(19) United States
(12) Patent Application Publication
(10) Pub. No.: US 2005/0096531 A1
(43) Pub. Date: May 5, 2005

(54) Magnetic field measurement apparatus

(76) Inventors: Mitsuru Oonuma, Tokyo (JP); Kazunori Yanagisawa, Tokyo (JP); Daisuke Suzuki, Tokyo (JP); Yoshiaki Amano, Tokyo (JP)

Correspondence Address: ANTONELLI, TERRY, STOUT & KRAUS, LLP, 1300 NORTH SEVENTEENTH STREET, SUITE 1800, ARLINGTON, VA, 22209-9889, US

(21) Appl. No.: 10/975,640

(22) Filed: Oct. 29, 2004

(30) Foreign Application Priority Data
Oct. 31, 2003 (JP) 2003-371501

(51) Int. Cl. A61B 5/05

(52) U.S. Cl. 600/409; 5/601; 324/248

(57) ABSTRACT

A magnetic field measurement apparatus is downsized and excellent in the handling ability by the measurement engineer without imposing the person to be measured on the load or anxiety. The magnetic field measurement apparatus includes a bed part having a stage on which the person to be measured lies on its top face, and a pipe-like magnetic shield room located on a floor in a predetermined positional relationship with the bed part. The bed part includes a measurement department move mechanism part that moves a measurement part disposed on one end side of the stage in the longitudinal direction to a center side of the stage, and a stage move mechanism part that moves the measurement part that has moved to the center side of the stage to an interior of the magnetic shield room together with the stage.

### CLAIM OF PRIORITY

The present invention claims priority from Japanese application JP 2003-371501 filed on Oct. 31, 2003, the content of which is hereby incorporated by reference into this application.

### FIELD OF THE INVENTION

The present invention relates to a downsized magnetic field measurement apparatus that measures a magnetic field developed from an object to be measured, for example, a slight magnetic field developed from a heart, brains, etc. of a living body by using a flux meter having a high-sensitive superconducting quantum interface device (SQUID).

### BACKGROUND OF THE INVENTION

Conventionally, a magnetic field measurement apparatus using a SQUID has been employed for measurement of a magnetic field developed from the brains or heart of a living body (hereinafter referred to as “living body magnetism”). In the measurement of a slight magnetic field such as a living body magnetism, it is necessary to attenuate an extrinsic magnetic field that is mixed in the magnetic field measurement apparatus to 80 dB to 100 dB (decibel) or higher. The extrinsic magnetic field is developed in such a manner that a magnetic noise that is derived from an electric feeder line, a traveling electric train, an automobile or the like is mixed into the magnetic field measurement apparatus through a commercial power supply.

The conventional living body magnetism measurement apparatus is located in the interior of a magnetic shield room using a ferromagnetic substance such as permalloy, and conducts measurement in an environment where an extrinsic magnetic field is shielded. The magnetic shield room using the ferromagnetic substance such as permalloy is expensive, large in size and heavy in weight, and therefore a medical agency where such a magnetic shield room is locatable is limited. A living body magnetic measurement using a magnetic shield that is lighter in weight and smaller in size and simple is desired so that the magnetic shield room is readily located even at a small place.

To meet the above requirement, there has been proposed a ferromagnetic substance such as permalloy is shaped in a pipe, and a plurality of measurement flux meters (hereinafter referred to as “sensors”) are arranged within a pipe-like opening part (see Japanese Patent Laid-Open No. 2000-175874). Then, a person to be measured who lies on a bed or the like is inserted into the pipe-like opening part, to thereby make it possible to perform measurement by means of the sensors. In the proposal, a direction perpendicular to a detection surface that is made up of the plurality of sensors is so arranged as to be orthogonal to a main axis (x axis) of the cylinder. As a result, the measurement can be performed with the elimination of an influence of the extrinsic magnetic field. However, in the proposal, a principle using the magnetic shield room that is formed in the cylinder has been proposed, but no practical proposal is made.

On the other hand, in order to facilitate the measurement of the person to be measured in the above magnetic shield room, there has been proposed a sensor-equipped bed in which a sensor is movably attached to the bed, and when the person to be measured lies on the bed, the detection surface can be positioned without exerting a load on the person to be measured (see Japanese Patent Laid-Open No. 2002-136492).

According to the above proposal, since a slight magnetic field can be measured without any provision of the big shield room, the proposal can greatly contribute to the downsized magnetic field measurement apparatus. However, the above proposal does not specifically disclose how the person to be measured who has lied on the bed is inserted into or removes from the pipe-like magnetic shield room. The above apparatus faces serious problems in practical use because it is required that the person to be measured does not suffer from a load or anxiety even in various circumstances, and an measurement engineer can readily perform work.

As one method of solving the above problem, there has been proposed that a detector is movably attached to the bed. However, the conventional example does not consider the application of work to the pipe-like magnetic shield room because it is assumed that work is conducted in the large magnetic shield room.

The present invention has been made in view of the above circumstances, and therefore an object of the present invention is to provide a downsized magnetic field measurement apparatus which is excellent in the handling ability by the measurement engineer without imposing the person to be measured on the load or anxiety.

### SUMMARY OF THE INVENTION

In order to achieve the above object, according to the present invention, there is provided a magnetic field measurement apparatus that comprises a bed part having a stage for putting the person to be measured on a top surface of the stage, and a pipe-like magnetic shield room located on a floor surface in a predetermined positional relationship with the bed part. The bed part includes a measurement department move mechanism part that moves a measurement department disposed on one end side of the stage in a longitudinal direction of the stage to a central side of the stage, and a stage move mechanism part that moves the measurement department that has been moved to the central side of the stage to an interior of the magnetic shield room together with the stage.

According to the present invention, there is provided a downsized magnetic field measurement apparatus which is excellent in the handling ability by the measurement engineer without imposing the person to be measured on the load or anxiety.

### BRIEF DESCRIPTION OF THE DRAWINGS

FIG. 1 is an external view showing a magnetic field measurement apparatus in accordance with a first embodiment of the present invention;

FIG. 2A is a cross-sectional side view for explaining a measurement principle according to the first embodiment;

FIG. 2B is a cross-sectional front view for explaining the measurement principle according to the first embodiment;

FIG. 3 is a structural diagram showing parts of the apparatus according to the first embodiment;

FIG. 4 is a structural diagram showing parts of the apparatus according to the first embodiment;

FIG. 5 is an external view showing the magnetic field measurement apparatus according to the first embodiment;

FIG. 6A is a plan view showing a contour of a bed part according to the first embodiment;

FIG. 6B is a side view showing the contour of the bed part according to the first embodiment;

FIG. 6C is a front view showing the contour of the bed part according to the first embodiment;

FIG. 6D is a plan view showing an operation part of the bed part according to the first embodiment;

FIG. 7 is an external view showing a magnetic shield room measurement apparatus in accordance with the first embodiment of the present invention;

FIG. 8 is a structural diagram showing parts of the magnetic shield room in accordance with the first embodiment of the present invention;

FIG. 9A is a front view showing the magnetic shield room according to the first embodiment;

FIG. 9B is a right side view showing the magnetic shield room according to the first embodiment;

FIG. 9C is a plan view showing the magnetic shield room according to the first embodiment;

FIG. 9D is across-sectional plan view showing the magnetic shield room according to the first embodiment;

FIG. 9E is a cross-sectional side view showing the magnetic shield room according to the first embodiment;

FIG. 9F is a partially cross-sectional view showing the magnetic shield room according to the first embodiment;

FIG. 10 is a block diagram showing an operation control device according to the first embodiment;

FIG. 11A is an external view showing a state where the bed part is combined with the magnetic shield room according to the first embodiment;

FIG. 11B is a side view showing the state where the bed part is combined with the magnetic shield room according to the first embodiment;

FIG. 12A is a cross-sectional view for explaining the operation of a first state and a second state according to the first embodiment;

FIG. 12B is a cross-sectional view showing a third state according to the first embodiment;

FIG. 13 is an external view showing a use condition of a magnetic field measurement apparatus in accordance with a second embodiment of the present invention;

FIG. 14 is an external view showing a contour of a bed part according to the second embodiment;

FIG. 15A is a plan view showing the bed part according to the second embodiment;

FIG. 15B is a side view showing the bed part according to the second embodiment;

FIG. 15C is a front view showing the bed part according to the second embodiment;

FIG. 15D is a back view showing the bed part according to the second embodiment;

FIG. 16A is an external view showing a magnetic shield room according to the second embodiment;

FIG. 16B is a cross-sectional side view showing the magnetic shield room according to the second embodiment;

FIG. 16C is a front view showing the magnetic shield room according to the second embodiment;

FIG. 16D is a side view showing the magnetic shield room according to the second embodiment;

FIG. 16E is a cross-sectional plan view showing the magnetic shield room according to the second embodiment;

FIG. 16F is a plan view showing the magnetic shield room according to the second embodiment; and

FIG. 17 is an explanatory diagram showing a move mechanism in accordance with the second embodiment;

### DETAILED DESCRIPTION OF THE PREFERRED EMBODIMENTS

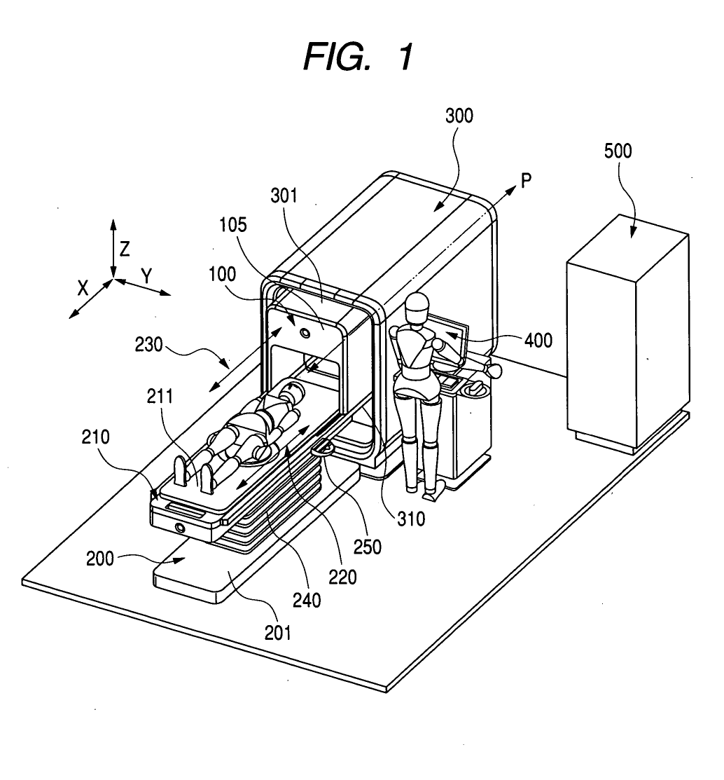

Now, a description will be given in more detail of a magnetic field measurement apparatus of the present invention with reference to FIGS. 1 to 17. Hereinafter, a heart magnetism measurement apparatus developed from a heart will be described with a living body as an object to be measured. However, the present invention is not limited to this example. For example, the present invention is also applicable to a measurement apparatus that detects magnetism such as presence or absence, or the amount of a magnetic substance contained in a normal object to be measured, or the distribution of the magnetic substance. The following disclosure shows only one embodiment of the present invention and does not limit the technical scope of the present invention. In addition, the same parts or functions are designated by like reference, and duplicate description will be omitted.

### First Embodiment

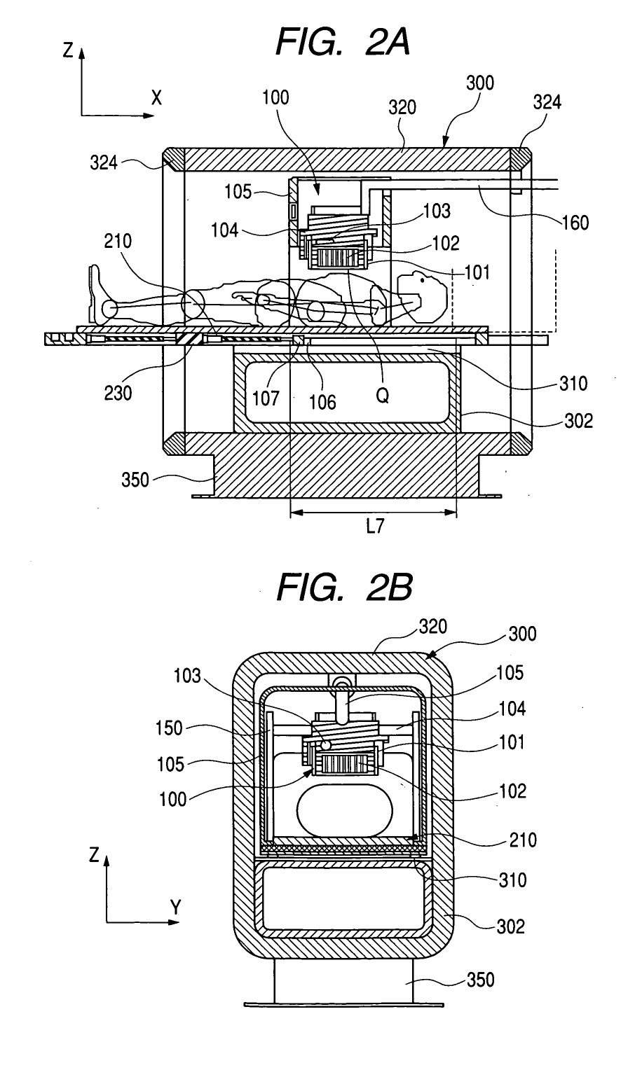

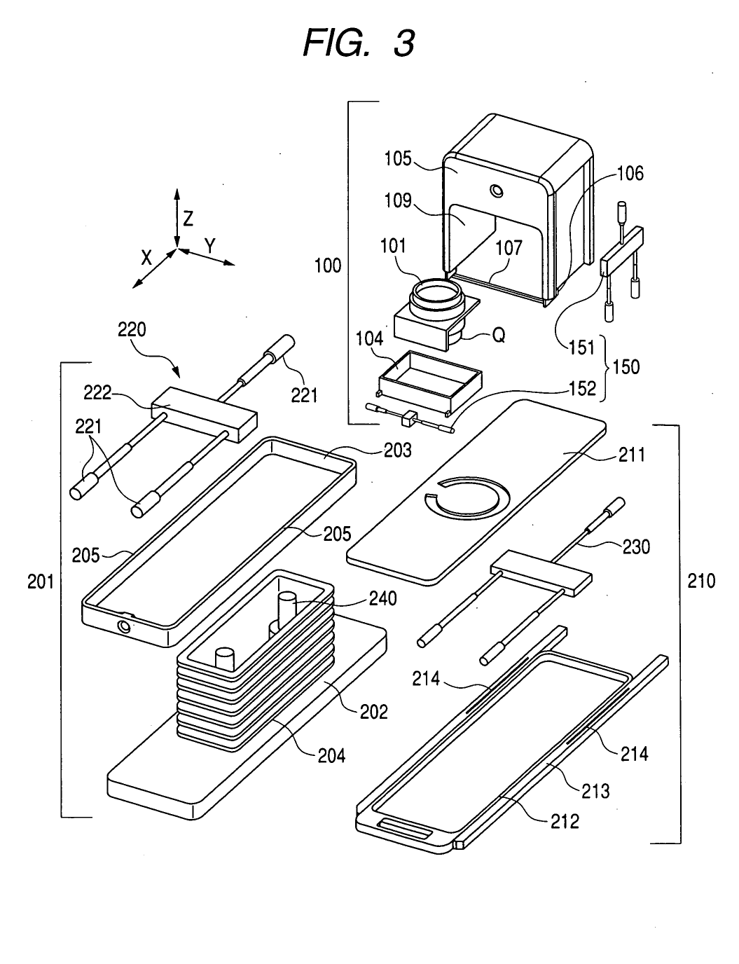

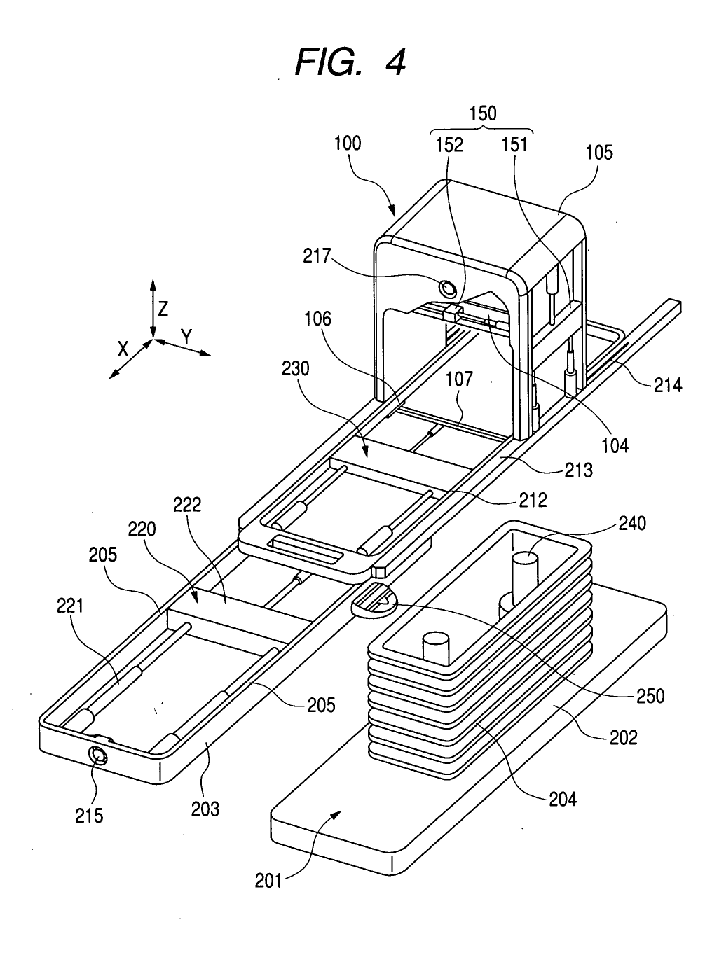

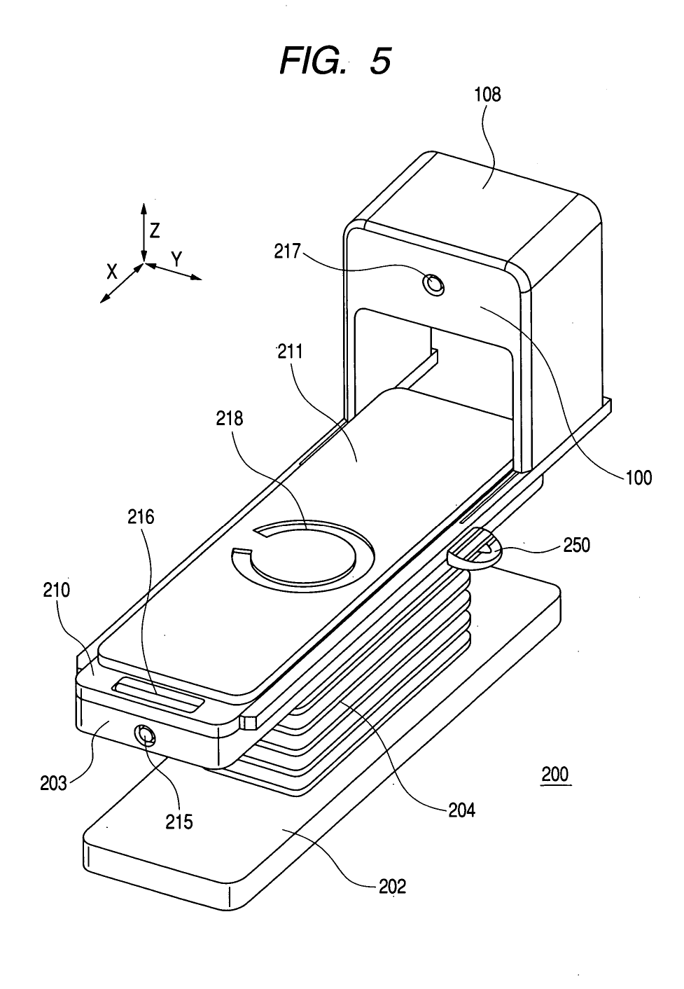

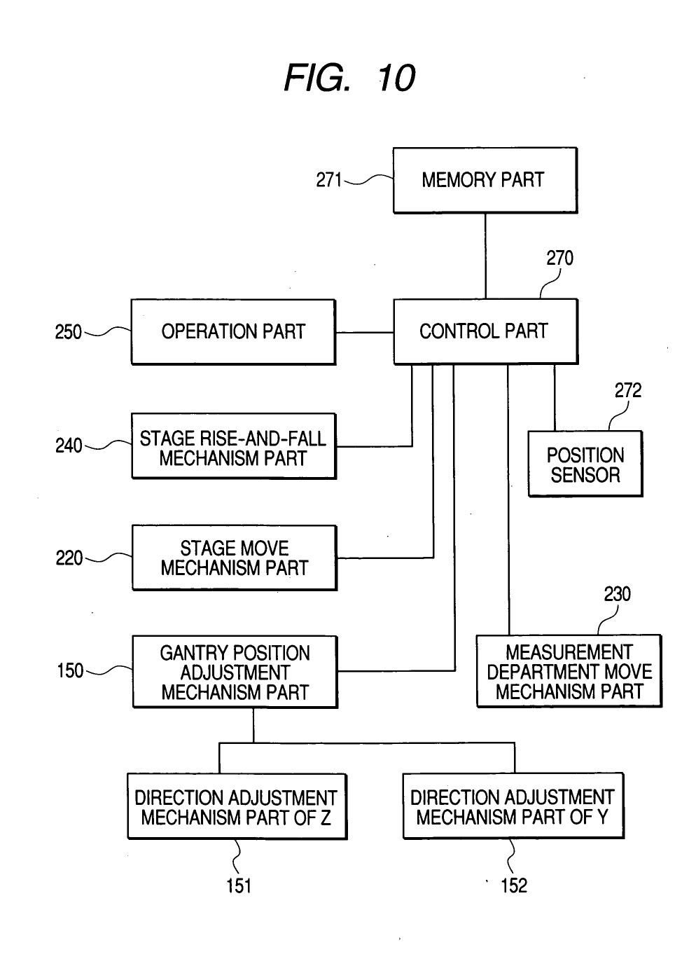

FIGS. 1 to 12 show a heart magnetism measurement apparatus in accordance with a first embodiment of the present invention. FIG. 1 is an external view of the apparatus, and FIGS. 2A and 2B are views of a measurement principle. FIGS. 3 and 4 are structural diagrams of parts of the apparatus. FIGS. 5 to 9F are external views of the apparatus, FIG. 10 is a diagram of an operation control device, and FIG. 1A to 12B are explanatory diagrams of the operation.

First, the outlined structure of a heart magnetism measurement apparatus according to this embodiment will be described with reference to FIG. 1.

Referring to FIG. 1, the heart magnetism measurement apparatus includes a bed part 200 having a measurement part 100 with a magnetic flux meter (hereinafter referred to as “sensor”) using an SQUID (superconducting quantum interface device), a magnetic shield room 300 allowing the measurement part 100 to indwell therein and removing the extrinsic magnetic field at the time of measurement, a data collection analysis equipment 400 that conducts the adjustment of magnetic field data acquired from the measurement part 100, setting of measurement conditions, various analysis or the like, and a magnetic field measurement drive 500 that analyzes the magnetic field data collected from the sensor.

The bed part 200 and the magnetic shield room 300 are located on a floor in a predetermined positional relationship. The data collection analysis equipment 400 and the magnetic field measurement drive 500 are appropriately located in the vicinity of the magnetic shield room 300. In this embodiment, in order to improve the operationality of the measurement engineer, the data collection analysis equipment 400 is so disposed as to be adjacent to the magnetic shield room 300.

The bed part 200 includes a basis 201 fixed onto the floor, a stage 210 disposed on an upper surface of the basis 201, and the measurement part 100 disposed on an upper part of one end side of the stage 210 in a longitudinal direction thereof. In addition, the stage 210 is movably disposed in the longitudinal direction of the stage 210 through a stage move mechanism part 220. The measurement part 100 is movably fitted to the stage 210 through the measurement department move mechanism part 230 in the longitudinal direction thereof. In FIG. 1, the stage move mechanism part 220 and the measurement department move mechanism part 230 are shown in the move direction, and their details will be described with reference to FIGS. 3 and 4.

In this example, the longitudinal direction of the stage 210 is defined as an X direction, a horizontal direction of the stage 210 which is orthogonal to the X direction is defined as a Y direction, and a vertical direction of the stage 210 is defined as a Z direction. With the above definitions, the following description will be made.

On the other hand, the magnetic shield room 300 has a pipe-like appearance and is located such that a main axis P of the pipe-like opening part 301 coincides with the X direction. In addition, the pipe-like opening part 301 has a size for indwelling the stage 210 having the measurement part 100 therein, and a stage support means 310 for supporting one end of the stage 210.

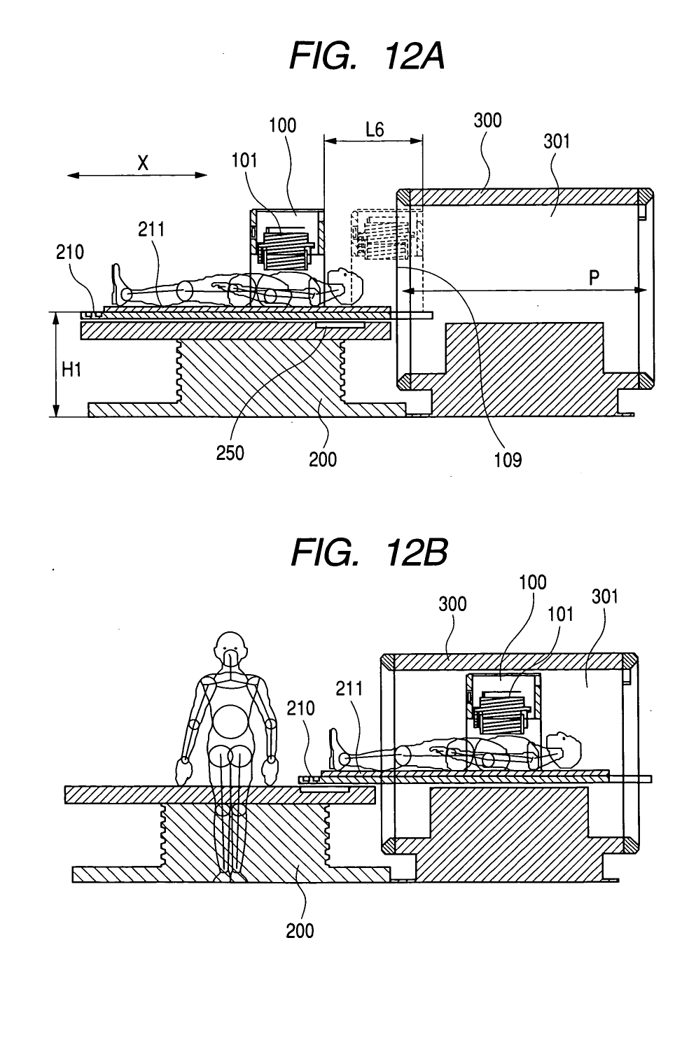

As described above, the heart magnetism measurement apparatus has first and second move mechanism parts consisting of the stage move mechanism part 220 and the measurement department move mechanism part 230. With this structure, the heart magnetism measurement apparatus can be readily put into an open first state where a person to be measured lies on the stage 210 (a state shown in FIG. 1), a second state where the measurement engineer applies the measurement part 100 to an affected part of the person to be measurement (a state shown in FIG. 12), and a third state where the extrinsic magnetic field is removed, and measurement can be conducted (a state shown in FIGS. 2A and 2B).

In order to obtain the above advantage, one feature of this embodiment resides in that a retreat position of the measurement part 100 is provided on one end side of the stage 210 in the longitudinal direction thereof (magnetic shield room 300 side), and the measurement part 100 is so disposed as to be movable along the X direction. That is, according to this embodiment, in the first state where the person to be measured lies on the stage 210, the measurement part 100 retreats into one end side of the stage 210 in the longitudinal direction thereof. Therefore, the periphery of the stage 210 on which the person to be measured lies comes to an open space. For that reason, the person to be measured can lie on the stage 210 without uncomfortable feeling, and moreover a care of the person to be measured by the measurement engineer can be readily conducted.

In this embodiment, the measurement part 100 is moved through the measurement department move mechanism part 230 to a measurement area of the person to be measured who lies on the stage 210, thereby making it possible to easily change from the first state to the second state.

In this embodiment, since the area to be measured is a heart, the person to be measured lies back on a bed 211 disposed on the stage 210 with his head positioned at the measurement part 100 side. Since the measurement part 100 has a tunnel-like configuration and is supported by both sides of the stage 210, the measurement part 100 is allowed to pass through the head of the person to be measured so as to move to the heart which is the area to be measured by the most direct way. Accordingly, in the second state where the measurement part 100 is moved toward the center of the most direction way, the measurement engineer can position the measurement part 100 to the heart of the person to be measured who lies on the stage 210 in a space opened to the exterior of the magnetic shield room 300, and further can position the sensor. In addition, since the movement distance of the measurement part 100 by the measurement department move mechanical part 230 can be shortened, this embodiment can contribute to the downsized apparatus.

Also, in this embodiment, the measurement department move mechanism part 230 is so arranged as to overhang outward from one end of the bed 211 on which the person to be measured lies when the measurement part 100 is moved to the outmost end side of the stage 210 in the longitudinal direction thereof. Then, the measurement department move mechanism part 230 operates. With this structure, a length of the stage 210 in the longitudinal direction thereof can be suppressed from being longer than the length as required.

In addition, the measurement part 100 has its lateral width set to a size that falls within a projected area of the stage 210, when viewed from above, and also set to be movable within the projected area. As a result, the safety of the movement of the measurement part 100 can be improved without enlarging the opening part 301 of the magnetic shield room 300.

Also, another feature of the heart magnetism measurement apparatus according to this embodiment resides in that the provision of the stage move mechanical part 220 makes it possible to move the stage 210 toward the direction of the retreat position of the measurement part 100. As a result, the measurement part 100 that keeps the positional relationship between the person to be measured and the measurement part 100 positioned at a predetermined position of the person to be measured is inserted into the magnetic shield room 300 up to a predetermined position, thereby making it possible to change from the second state to the third state. Moreover, since the stage support mechanism 310 that supports one end of the stage 210 to be inserted is disposed within the opening part 301 of the magnetic shield room 300, the stage 210 can be supported by the stage move mechanism part 220 and the stage support mechanism 310 in the third state where one end of the stage 210 is pulled out. Therefore, this embodiment can contribute to the downsized stage move mechanical part 220.

Also, still another feature of the hear magnetism measurement apparatus according to this embodiment resides in that the bed part 200 and the magnetic shield room 300 can be located in such a manner that the measurement part 100 is received in the opening part 310 of the magnetic shield room 300 in the first state. With this structure, since an installation interval between the bed part 200 and the magnetic shield room 300 which are disposed linearly can be shortened, the installation property can be improved. In addition, since a part of the measurement part 100 can be shielded from the person to be measured, the feeling of pressure of the person to be measured by the measurement part 100 can be reduced.

Also, yet still another feature of the heart magnetism measurement apparatus according to this embodiment resides in that drive means for the stage move mechanism part 220, the measurement department move mechanism part 230, and a Gantry position adjustment mechanism part 150 (which will be described with reference to FIGS. 3 and 4) and a stage rise-and-fall mechanism part 240 (which will be described with reference to FIGS. 3 and 4) within the measurement part 100 are automated by a move mechanism part that does not develop a magnetic field, and their operation switches are concentratedly disposed at one place. In this embodiment, a hydraulic mechanism is applied to reduce an influence of the magnetic field. However, another drive means that does not develop the magnetic field may be applied.

In addition, in this embodiment, an operation part 250 is arranged at one side of the stage 210 substantially in the center of the stage 210 in the X direction thereof. Since this position is in the vicinity of a hip position of the person to be measured, the measurement engineer readily helps the person to be measured who gets on and off the stage 210. Also, the measurement engineer easily observes the state of the measurement part 100 that passes through an upper area of the person to be measured who lies on the stage 210, and can visually position the sensor to the heart.

In addition, in this embodiment, various sensors are provided in the respective move mechanism parts from the viewpoint of safety.

Hereinafter, the heart magnetism measurement apparatus according to this embodiment will be described in more detail with reference to FIGS. 2A to 12B.

First, the measurement principle using the pipe-like magnetic shield room 300 will be described with reference to FIGS. 2A and 2B. FIGS. 2A and 2B are cross-sectional views showing the third state. FIG. 2A is a longitudinal cross-sectional view thereof, and FIG. 2B is a horizontal cross-sectional view thereof.

Referring to FIGS. 2A and 2B, in the third state, the stage 210 on which the person to be measured lies with his head held by a pillow and the measurement part 100 positioned at the heart of the person to be measured are disposed in the interior of the magnetic shield room 300. A cooling container 101 is disposed in the measurement part 100, and a plurality of sensors 102 and a 1st sensor for reference 103 are disposed on a inner bottom of the cooling container 101 and cooled with a refrigerant liquid filled in the cooling container 101. In this embodiment, the sensors (flux meters) made of a high-temperature superconductor which can operate at a temperature of liquid nitrogen are used, and liquid nitrogen is introduced into the cooling container 101 to cool the sensors 102 and 103. The cooling container 101 is held by a gantry 104 having a positioning mechanism.

The position of the gantry 104 that fixes the cooling container 101 is adjusted by a gantry position adjustment mechanism part 150 having the direction move mechanisms of Y and Z. In this embodiment, the gantry position adjustment part 150 has a hydraulic mechanism that develops no magnetic field so as to adjust the position of the gantry 104 by the hydraulic mechanism.

The stage 210 and units such as the measurement part 100 which are disposed in the interior of the magnetic shield room 300 are made of nonmagnetic material such as FRP (fiber reinforced plastics) or aluminum.

The detection coils of the plurality of sensors (flux meters for measurement) 102 are arranged on the same measurement plane Q that is in parallel with an X-Y plane perpendicular to the direction of Z, and the detection coil plane is perpendicular to the direction of Z. The sensors 102 detect the magnetic field component in the direction of Z. The 1st sensor for reference 103 is provided for detecting the X direction component of the extrinsic magnetic field, and disposed in the vicinity of the sensor 102 so that the detection direction of the magnetic field becomes the direction of X.

The drive operation of the plurality of sensors 102 and the 1st sensor for reference 103 is controlled by the magnetic field measurement drive 500 that is connected through a signal line 160 to the sensors 102 and the 1st sensor for reference 103. In the magnetic field measurement drive 500, an analog signal corresponding to the magnitude of a magnetic field detected by each of the sensors is processed by an analog signal processing circuit including an amplifier, a band-pass filter and a notch filter.

The signal line 160 is formed of a plurality of cables consisting of a bundle of cables that transmit a detected signal from the sensors 102 and the 1st sensor for reference 103 to the magnetic field measurement drive 500, and a bundle of cables for allowing a bias current, a feedback current and a heater current to flow into the sensors 102 and the 1st sensor for reference 103 from the magnetic field measurement drive 500.

An output of the magnetic field measurement drive 500 is converted into a digital signal and taken in the data collection analysis equipment 400. The data collection analysis equipment 400 executes various signal processing with respect to the take-in signals.

The extrinsic magnetic field includes three components of the X, Y and Z directions. Since the shield performance of the pipe-like magnetic shield room 300 is excellent in the Z and Y directions perpendicular to the X direction shown in FIG. 1, the components of the extrinsic magnetic field in the directions of Z and Y are greatly attenuated in the interior of the pipe-like magnetic shield room 300.

On the other hand, the components of the extrinsic magnetic field in the directions of X and Y are orthogonal to the magnetic field detection direction of the sensors (SQUID flux meters) 102. Most of those components are not detected by the sensors 102. Accordingly, the extrinsic magnetic field in the direction of Y which is shielded by the magnetic shield room 300 and low in the sensitivity of the sensors (SQUID flux meters) 102 can be ignored in practical use.

Most of the component of the extrinsic magnetic field in the direction of Z can be cancelled by calculating a difference between the output signals from the adjacent sensors 102. The difference can be calculated after the data is taken in the data collection analysis equipment 400.

The data collection analysis equipment 400 conducts arithmetic operation that corrects the component of the extrinsic magnetic field in the direction of X by subtracting a value obtained by multiplying a measurement signal from the 1st sensor for reference 103 by a predetermined coefficient from the difference of the output signals from the adjacent sensors 102, and obtains a first measurement signal that is reduced in an influence of the extrinsic magnetic field.

As described above, in this embodiment, the provision of the above structure makes it possible that the measurement conducted within the room that has been shielded from magnetism conventionally is conducted even in a place having no large magnetic shield facility. Therefore, the facility costs can be remarkably reduced, and the installation space of the apparatus can be reduced in size. Moreover, in this embodiment, since the person to be measured is measured within the pipe-like magnetic shield room 300, a feeling of closure or a feeling of insecurity of the person to be measured is reduced. In addition, since the measurement engineer exists adjacent to the downsized pipe-like magnetic shield room 300, the feeling of insecurity of the person to be measured is reduced.

Subsequently, the parts structure of the bed part will be described with reference to FIGS. 3 and 4. FIG. 3 is an expansion plan showing the parts of the bed part, and FIG. 4 is a structural diagram showing the parts.

Referring to FIGS. 3 and 4, as described above, the bed part 200 is made up of the basis 201, the stage 210 and the measurement part 100. In this embodiment, the basis 201 includes a foot 202 that is fixed to the floor, a stage support part 203, a stage rise-and-fall mechanism part 240 that is disposed between the foot 202 and the stage support part 203, an accordion-like cover 204 that covers the stage rise-and-fall mechanism part 240, and the stage move mechanism part 220 that is fitted to the stage support part 203.

The stage support part 203 has a box-like configuration that is thin in the direction of Z and opened upward, and the stage move mechanism part 220 is disposed in an opened space of the stage support part 203. The stage support part 203 has support rails 205 at both sides thereof in the direction of X. The stage 210 is fitted to the stage support part 203 so as to be movable through slide rails not shown which support the support rails 205 from both sides thereof and to cover an upper part of the stage support part 203. Also, the operation part 250 is disposed at one side of the stage support part 203 in the center thereof.

The stage move mechanism part 220 is made up of one or plural hydraulic cylinders. Because the sensors 102 using the SQUID measures a slight living body magnetic field, it is impossible to employ the move mechanism part such as a motor which develops the magnetic field. In this embodiment, all of the move mechanisms including the stage move mechanism part 220 have the hydraulic cylinder as in the stage move mechanism part 220, thereby realizing the automation of the move mechanism parts.

The stage move mechanism part 220 according to this embodiment uses antenna-like cylinders 221 that expand and contract at multistage, and is arranged randem by the intervention of an intermediate member 222 therebetween in such a manner that a longitudinal direction of the cylinders 221 coincide with the X direction. Then, two of those cylinders 221 are arranged in parallel at one end side of the stage move mechanism part 220, and one cylinder 221 is arranged at another end side. In addition, the cylinders 221 at one end side of the stage move mechanism part 220 are fixed to one end side of the stage support part 203 whereas the cylinder 221 at another end side thereof is fixed to the stage 210. Also, the intermediate member 222 is disposed movably along the X direction while both sides of the intermediate member 222 are guided by inner walls of the stage support part 203.

An oil feeder that feeds a hydraulic pressure to the cylinders 221 develop a magnetic field, and is therefore disposed outside of the measurement room.

As described above, in this embodiment, since the cylinder 221 is arranged randem, it is possible to suppress the size (diameter) of the cylinders 221 and increase a movable distance of the stage 210. Also, the adoption of the multistage cylinder makes it possible to further increase the movable distance of the stage 210. Since the cylinders 221 are coupled to each other randem through the intermediate member 222, and both sides of the intermediate member 222 are movably supported, the strength of the stage move mechanism part 220 can be enhanced. Also, since the stage move mechanism part 220 can be thinned, the thin stage support part 203 can be realized. In addition, with the provision of the intermediate member 222 and the pair of cylinders 221 makes it possible to reduce chatter of the stage 210 during the movement.

Also, the stage rise-and-fall mechanism part 240 can be made up of a diversity of mechanisms. In this embodiment, the hydraulic pressure system is adopted. With this structure, the magnetic field is suppressed from occurring, and the person to be measured is easy to lie on the stage 210. Also, the level adjustment for reducing a load on a hip of the measurement engineer can be performed. The stage rise-and-fall mechanism part 240 is covered with an accordion cover 204 taking the safety and design into consideration.

The stage 210 is made up of a frame component 212, the bed 211 and the measurement department move mechanism part 230. The frame component 212 is shaped in a frame, and measurement department support rails 213 that move the measurement part 100 are fitted to both sides of the frame component 212 in the X direction.

The measurement part support rails 213 overhangs from one end side of the frame component 212 in the X direction thereof (magnetic shield room 300 side). Support slots 214 are formed from the overhanging part side toward another end side. In this embodiment, the measurement part 100 is movably supported on the upper part of the measurement department support rails 213. In this situation, projections 106 are provided on a slide face of the measurement part 100 side. The projections 106 penetrate the support slots 214 in the Z direction so that the measurement part 100 is fitted to the measurement department support rails 213, thereby making it possible to move the measurement part 100 in the X direction without derailing from the measurement department support rails 213.

The bed 211 has a cushioning mat attached onto the top face of a plate member so that the person to be measured can lie on the bed 211 without uncomfortable feeling. The stage 210 forms a thin space in association with the frame component 212 and the bed 211, and the thin measurement department move mechanism part 230 having the same structure as that of the stage move mechanism part 220 is disposed in the space. One end of the measurement department move mechanism part 230 is fitted to one end side of the frame component 212, and other end of the measurement department move mechanism part 230 is fitted to the measurement part 100.

The measurement part 100 is made up of a measurement main part 105 that is shaped in a gate when being viewed from the X direction, a gantry 104 that is arranged in the measurement part 100, the cooling container 101 disposed on the gantry 104, and a gantry position adjustment mechanism part 150 that adjusts the position of the gantry part 104.

The measurement main part 105 has a gate-like appearance having a top face and both side faces formed by a top plate and side plates, and a front face and a rear face formed by upper flat plates and lower opening parts 109. With this structure, when the gantry 104 is retreated to the uppermost part, the gantry 104 is substantially defiladed to improve the uncomfortable feeling of the person to be measured and the design. Then, the pair of side plates that extend downward are fitted to the pair of measurement department support rails 213, and movably support the entire measurement part 100. In this situation, since the opening part 109 that penetrates in the X direction is defined between the pair of side plates, the measurement part 100 can move in the X direction so that the person that lies on the stage 210 is inserted into the opening part 109.

Also, each of the projections 106 is disposed on one end side of the lower end part of each of the side plate. Each of the projections 106 is fitted to each of the measurement department support rails 213 so as to penetrate each of the support slots 214 of the measurement department support rails 213. The projections 106 are coupled to each other by means of a connection stick 107. As shown in FIGS. 2A and 2B, one end of the measurement department move mechanism part 230 is fitted to the connection stick 107. With this structure, a length L7 of the support slots 214 are so shortened as to improve the strength of the measurement department support rails 213.

Returning to FIGS. 3 and 4, the gantry 104 has a box-like appearance for receiving the cooling container 101, and is movably supported to the measurement main part 105 by means of the gantry position adjustment mechanism part 150. The gantry position adjustment mechanism part 150 is made up of a direction adjustment mechanism part of Z 151 which moves the cooling container 101 in the direction of Z, and a direction adjustment mechanism part of Y 152 which moves the cooling container 101 in the direction of Y. The direction adjustment mechanism part of Z 151 and the direction adjustment mechanism part of Y 152 have the same structure as that of the stage move mechanism part 220. The direction adjustment mechanism part of Z 151 is disposed on an inner wall face of the side plates so as to move the gantry 104 in the direction of Z. Also, the direction adjustment mechanism part of Y 152 is fitted to the gantry 104 so as to move the cooling container 101 in the direction of Y. The gantry position adjustment mechanism part 150 having those two move mechanism parts makes it possible to position the bottom face of the cooling container 101 which is a measurement plane Q to a predetermined position of the person to be measured.

In order to position the measurement plane Q to an affected area of the person to be measured who lies on the stage 210, it is necessary to adjust the direction of X of the stage 210, the directions of Y and Z which are orthogonal to the direction of X, respectively. In this embodiment, the adjustment in the direction of X is conducted by the measurement department move mechanism part 230, and the adjustments in the directions of Y and Z are conducted by the gantry position adjustment mechanism part 150. This allows the number of positioning move mechanism parts to be reduced. It is needless to say that a move mechanism part that conducts the adjustment in the direction of X may be provided in the gantry position adjustment mechanism part 150. In this case, it is preferable that the measurement department move mechanism part 230 can move the measurement plane Q to a coarse position, and the measurement department move mechanism part 230 conducts fine adjustment.

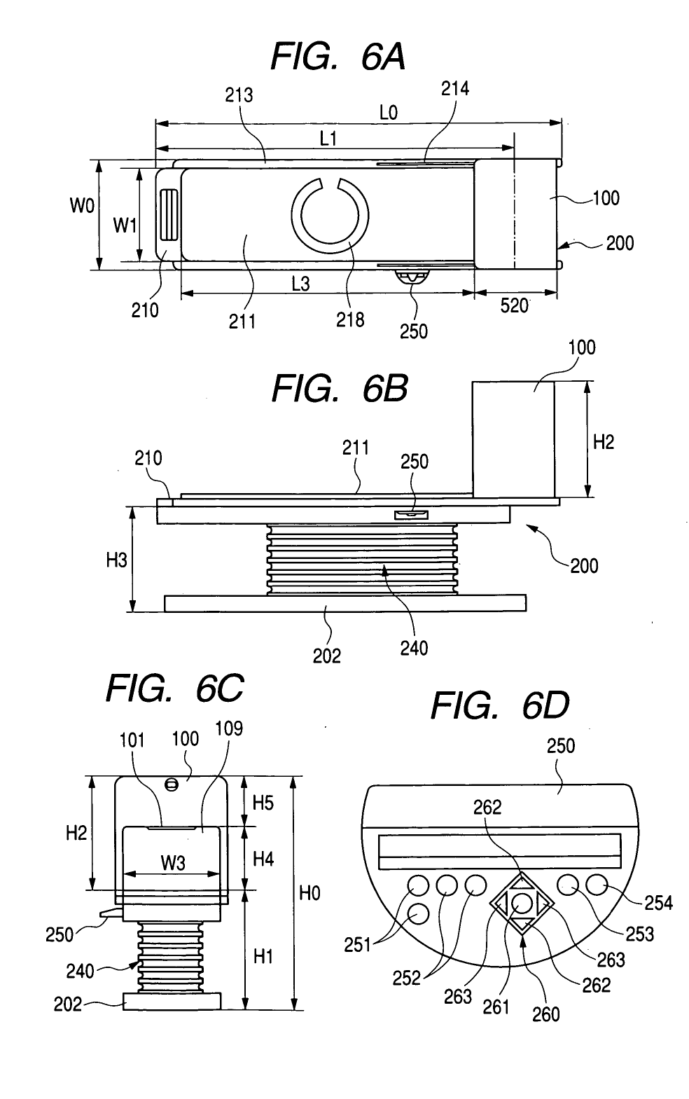

Subsequently, the exterior structure and dimensional system of the bed part will be described with reference to FIGS. 5 and 6D. FIG. 5 is a perspective view showing the appearance of the bed part in the first state, and FIGS. 6A to 6D are diagrams showing the bed part in the first state, in which FIG. 6A is a top view thereof, FIG. 6B is a front view thereof, FIG. 6C is a right side view thereof, and FIG. 6D is an enlarged view showing the operation part.

First, referring to FIG. 5, in the bed part 200 according to this embodiment, the stage support part 203, the stage 210 that is fitted on the upper part of the stage support part 203 and the foot 202 have substantially the same rectangular configurations slender in the direction of X. With this structure, two thin rectangular plates are connected to each other by means of the accordion cover 204 that is smaller than those thin rectangular plates by one size, to thereby downsize the bed part 200. Moreover, the measurement part 100 that is disposed on one side of the stage 210 in the longitudinal direction thereof is shaped in a box having the same width as that of the stage 210, thereby making the exterior configuration of the entire bed part 200 in the longitudinal direction thereof simple.

An end part of the stage support part 203 that overhangs in the longitudinal direction is provided with a 1st emergency stop button 215 that brings the move mechanism part to a free state, and a handle 216 for moving the stage 210 manually is disposed on an end part of the stage 210 which is adjacent to the 1st emergency stop button 215. As a result, when an emergency occurs in the third state, the 1st emergency stop button 215 is operated to bring the move mechanism part to the free state, and the stage 210 can be immediately pulled out of the magnetic shield room 300 through the handle 216. Moreover, since the 1st emergency stop button 215 is disposed on a vertical face of the end part of the stage support part 203, the erroneous operation becomes hard. Also, since the handle 216 is largely provided on the top face of the end part of the stage 210, the pull-out operation is comfortable.

Also, in this embodiment, a 2nd emergency stop button 217 is disposed on a vertical face of the measurement part 100 at the operation part 250 side. With this structure, the measurement engineer that operates the operation part 250 and the data collection analysis equipment 400 can immediately reach the 2nd emergency stop button 217 and bring the move mechanism part to the free state. Also, the position of the vertical face where the 2nd emergency stop button 217 is arranged is a position where the erroneous operation is made hard.

Also, in this embodiment, a mark 218 is added to the center of the bed part 211, thereby making it possible that the person to be measured can recognize a position at which he lies. In this embodiment, the mark 218 is provided substantially in the center of the bed part 211 in the direction of X as a target for the position of his hip when the person to be measured lies on the bed part 211. The mark 218 is disposed by embedding a cushioning component having a color different from that of the cushioning mat that covers the upper part of the bed part 211 in the mat. Moreover, the mark 218 is shaped in C so as to influence the person to be measured to get on the stage 210 from an open side of the C-shape.

In addition to the above structure, according to this embodiment, the operation part 250 is disposed at a side opposite to the open side of the C-shape. With this structure, the sides at which the measurement engineer and the person to be measured are positioned, respectively, are clarified to reduce the erroneous operation.

Also, a maintenance cover 108 is disposed on the top face of the measurement part 100. The maintenance cover 108 is to restock the cooling container 101 with a refrigerant liquid. In this embodiment, because the top face of the measurement part 100 is flat, the maintenance cover 108 can be provided with a simple structure.

Referring to FIGS. 6A to 6D, a height H1 of from the floor to the top face of the bed part 211 is set to 715 mm to improve the workability of the measurement engineer. In general, when the workability of the vertical posture is set to a range of from 600 mm to 900 mm, most of the workability of the measurement engineer can be covered. In particular, in this embodiment, since the person to be measured lies on the bed part 211, the height is set so that care for the person to be measured is satisfactorily taken. In this embodiment, the height of the magnetic shield room 300 is set with the height H1 to the top face of the bed part 211 as a reference value.

The stage 210 has a size that is 700 mm in lateral width W0 and 2150 mm in length L1. In this embodiment, since the handle 216 is overlapped with one end side of the stage 210, and the measurement part 100 is overlapped with another end side of the stage 210, the size of the bed part 211 when being viewed from the top face is 600 mm in the lateral width W1 and 1750 mm in L3. The size of L3 is slightly shorter in the length when the person to be measured having a standard body height is a reference. However, since the opening part 109 that is 425 mm in the height H4 is formed at the side of the person to be measured of the measurement part 100, the size when the person to be measured substantially lies on the bed part is included in the interior of the measurement part 100. The size of L3 is a size by which the head of the person to be measured is hardly abutted against the measurement part 100. In this embodiment, since the size of L3 is set to be slightly smaller, the installation space can be reduced. In addition, even if the person to be measured is taller, a measure can be made such that the pasterns are laid on the handle 216.

The bed part 211 is shaped in such a concave curved surface that a center of the bed part 211 is concave and both sides thereof are raised when being viewed from the X direction, and the person to be measured is easy to lie on the center of the bed part 211. Accordingly, since the person to be measured is influenced to lie back on a standard position of the bed part 211 according to the concave curved surface of the bed part 211 and the mark 218, the care of the person to be measured by the measurement engineer is easy, and the subsequent measurement work can be facilitated.

Also, since the measurement department support rails 213 that are 50 mm in the lateral width are disposed at both sides of the bed part 211, the measurement part can be stably supported. In addition, when the lateral width of the measurement department support rails 213 is 50 mm, the support slots 214 can be defined while the strength is maintained.

The measurement part 100 that is 725 mm in the height H2 is disposed on one side of the upper part of the stage 210. A height H0 of from the floor to the top face of the measurement part 100 is set to 1450 mm. Therefore, since the height H0 to the top face is set to be lower than the position of a standard adult eye line, the volume feeling of the overall apparatus can be reduced.

In addition, since the size of the measurement part 100 is set to be substantially the same as the lateral width W0 of the stage 210, the person to be measured does not feel the size of the apparatus. Moreover, since the overall measurement part 100 is boxy with rounded corners, the feeling of pressure and an awful feeling from which the person to be measured suffers can be reduced. In addition, the safety and the design at the time of moving the measurement part 100 are also improved.

Also, in FIG. 6C, the opening part 109 that is 600 mm in the lateral width W3 and 425 mm in the height H4 is formed at the lower part of the measurement part 100. When the opening part 109 is of this size, even a big person can be received in the opening part 109. In addition, a storage space that is hidden from the periphery and 310 mm in the height H5 is formed above the opening part 109. When the storage space is of this size, the cooling container 101 that is 300 mm in the diameter and 285 mm in the height can be substantially received in the storage space so as to be hidden from the person to be measured and the measurement engineer. Moreover, when the storage space has the lateral width W0, the cooling container 101 can be finely adjusted in the direction of Y.

On the other hand, the bed part 200 is located on the floor by the foot 202 having the same size as that of the stage 210. In this embodiment, in the first state shown in FIGS. 6A to 6C, because the measurement part 100 is retreated at one side of the stage 210, the stage rise-and-fall mechanism part 240 is disposed at the retreat position side of the measurement part 100. With this structure, the stable installation in the first state which is the normal state can be performed, and a lower limb space at the other end side opposite to the retreat position in the direction of X is broadened, thereby making the handling property and the impression of the downsized apparatus excellent.

Subsequently, in FIG. 6D, in this embodiment, the operation switches of the move mechanism part are concentrated at the operation part 250 which is disposed at the side surface of the stage support part 203. The operation part 250 is disposed in the middle of the mark 218 and the retreat position of the measurement part 100. This position is the best position for grasping the movements of the person to be measured and the entire apparatus and a position at which the measurement engineer is easy to address various changes in the conditions. Then, since the measurement engineer frequently traverses that position of the operation part 250, the operation part 250 is formed in a circular external configuration when being viewed from above taking the safety into consideration.

The operation part 250 includes a pair of up-and-down switches 251 of the stage up-and-down mechanism part 240, a pair of move switches 252 of the measurement department move mechanism part 230, an adjustment switch country 260 of the gantry position adjustment mechanical part 150, and a move switch 253 and a return switch 253 for driving the stage move mechanism part 220.

One of the up-and-down switches 251 is a down switch, and the other switch is an up switch. The stage 210 is returned to the reference height while the up switch continues to be depressed. One of the move switches 252 is a switch for pulling out the measurement part 100 from the retreat position, and the other switch is a switch for moving the measurement part 100 toward the retreat position.

The adjustment switch country 260 has a lock switch 261 arranged in the center thereof. A pair of rise-and-fall switches 262 for rising and falling the direction adjustment mechanism part of Z 151 are disposed at vertical positions by the intervention of the lock switch 261 when being viewed from the measurement engineer. A pair of move switches 263 that operate the direction adjustment mechanism part of Y are disposed at right and left positions. The lock switch 261 is operated to lock the gantry position adjustment mechanism part 150 and the measurement department move mechanism part 230 so as to fix the positional relationship of the measurement engineer and the sensors 102. The move switch 253 is a switch for moving the stage 210 to a predetermined position within the magnetic shield room 300 so as to bring the stage 210 to the third state. The return switch 253 is a switch for returning the stage 253 from the third state to the first state.

Those switches are arranged in the stated order of the up-and-down switch 251, the move switch 252, the adjustment switch country 260, the move switch 253 and the return switch 253 according to an operation procedure, to thereby reduce the erroneous operation and improve the operationality.

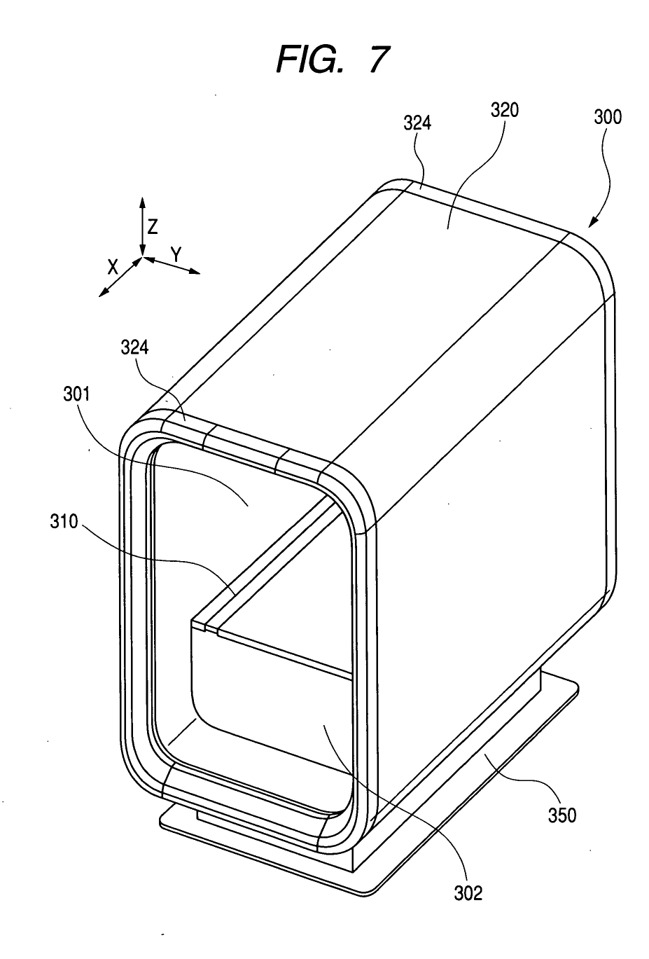

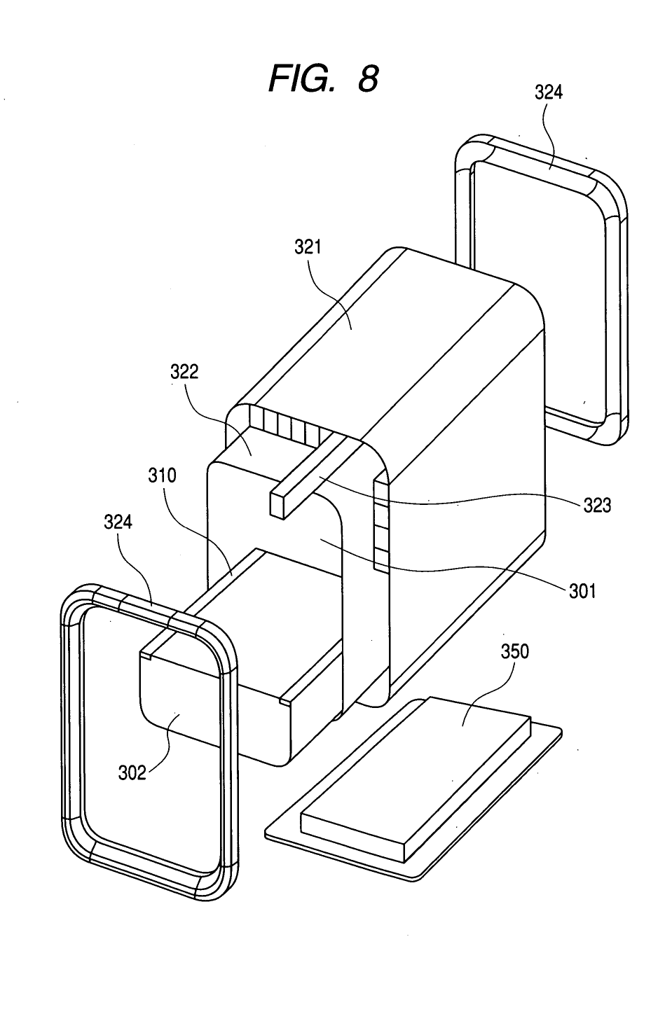

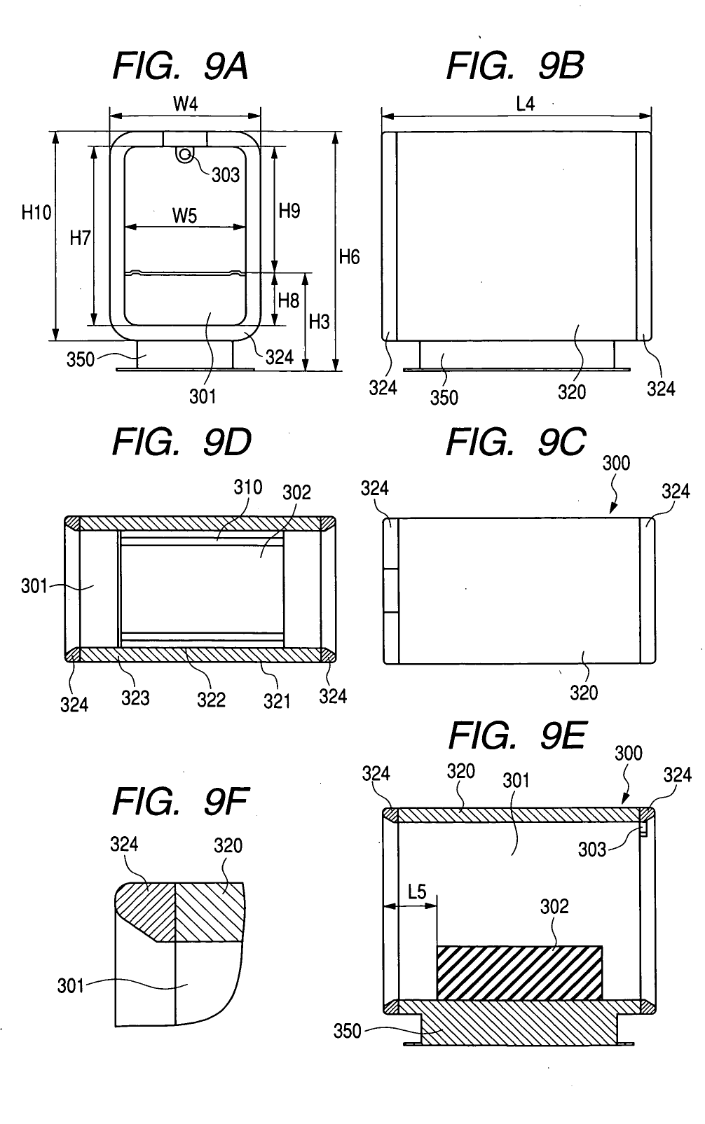

Subsequently, the magnetic shield room will be described in more detail with reference to FIGS. 7 to 9E. FIG. 7 is an external perspective view showing the magnetic shield room, FIG. 8 is a structural view showing the parts of the magnetic shield room, and FIGS. 9A to 9F are external views showing the magnetic shield room, in which FIG. 9A is a left side view thereof, FIG. 9B is a front view thereof, FIG. 9C is a plan view thereof, FIG. 9D is a horizontal cross-sectional view thereof, FIG. 9E is a vertical cross-sectional view thereof, and FIG. 9F is a partially enlarged cross-sectional view of the opening part.

First, the rough structure of the magnetic shield room 300 will be described with reference to FIG. 7. In this embodiment, the magnetic shield room 300 has a rectangular pipe-like external configuration with four rounded corners. The magnetic shield room 300 is made up of the pipe-like shield room main part 320, and the foot that supports the shield room main part 320 on the lower side thereof. The shield room main part 320 has a plinth part 302 within the pipe-like opening part 301 and the stage support means 310 on the top face of the plinth part 302. In this embodiment, the stage support means 310 is made up of a pair of support rails and supports support rails disposed on the bottom face of the stage 210. The plinth part 302 is located at a position apart slightly backward from the end of the opening part 301. This is because the measurement part 100 is received within the opening part 301 together with the stage 210 in the first state of the bed part 200 according to this embodiment. In the state where the measurement part 100 is received in the opening part 301, the stage 210 moves up and down by means of the stage rise-and-fall mechanism part 240. For that reason, the plinth part 302 is retreated backward so as not to impede the up and down movement of the stage 210. In other words, the magnetic shield room 300 serves as a retreat and receive part of the measurement part 100 in the first state in addition to the purpose of the magnetic shield for the original measurement.

The plinth part 302 supports one end of the stage 210 while the bed part 200 changes from the second state to the third state, and supports both ends of the stage 210 in the longitudinal direction thereof in the third state together with the stage move mechanism part 220. As a result, since a load of the stage move mechanism part 220 can be reduced, this structure can greatly contribute to the downsized stage move mechanism part 220.

Although being not shown, the pair of support rails of the stage support means 310 have corners at their ends slanted, and rollers are disposed on the ends of the support rails that are located on the bottom face of the stage 210. With this structure, when the support rails of the stage 210 come in contact with the pair of support rails of the stage support means 310 with the movement of the stage 210, the rollers are easy to run on the support rails of the stage support means 310 from the slanted faces thereof. As a result, the stage 210 can be smoothly moved.

Referring to FIG. 8, in this embodiment, the shield room main part 320 is made up of a pipe-type magnetically shielded outer side member 321, a pipe-type magnetically shielded inner side member 322 that is smaller than the magnetically shielded outer member 321 by one size, a plurality of adjustment component that adjusts a gap between the magnetically shielded inner member 322 and the magnetically shielded outer member 321, frame-like covers 324 that structure both ends of the shield room main part 320 in the direction of X, and the plinth part 302.

The magnetically shielded outer member 321 and the magnetically shielded inner member 322 are made of permalloy which is ferromagnetic substance. Since permalloy must be subjected to a heat treatment, the magnetically shielded outer member 321 and the magnetically shielded inner member 322 are uniquely formed in this embodiment. Also, in this embodiment, in order to enhance the efficiency of removing the extrinsic magnetic field and saving the weight, the shield room main part 320 has a double structure consisting of the magnetically shielded outer member 321 and the magnetically shielded inner member 322. Then, the plurality of adjustment components are disposed between the magnetically shielded outer member 321 and the magnetically shielded inner member 322 to adjust the positional relationship therebetween.

Also, components other than the magnetically shielded outer member 321 and the magnetically shielded inner member 322 are made of non-magnetic material such as FRP (fiber reinforced plastic) or aluminum.

In this embodiment, the part structure can make the assembling property excellent. For example, in assembling the magnetic shield room 300, the foot 350 is first fitted to the bottom face of the magnetically shielded outer member 321, and the plinth part 302 is fitted to the magnetically shielded inner member 322. Then, the magnetically shielded inner member 322 is attached to the interior of the magnetically shielded outer member 321 through the adjustment components 323. Thereafter, the covers 324 can be attached to both ends of the shield room main part 320.

In this embodiment, a structure is made such that the plinth part 302 is fitted to the magnetically shielded inner member 322. Alternatively, the former may be integrated with the latter. Also, it is not always necessary that the plinth part 302 has the size that blocks the lower part of the pipe-like opening part 301 as in this embodiment. Since it is necessary to provide only a function of holding the stage support means 310, the plinth part 302 may be replaced by projections to which the stage support means 310 is attached.

Then, referring to FIGS. 9A to 9F, in this embodiment, a height H6 of from the floor to the magnetic shield room 300 is set to 1600 mm, a height H10 of the shield room main part 320 is set to 1400 mm, a lateral width W4 is set to 1000 mm, and a depth L4 is set to 1800 mm. Also, a height H7 of the pipe-like opening part 301 is set to 1200 mm, and a lateral width W5 is set to 800 mm. In this embodiment, in order to reduce the overall volume feeling of the magnetic shield room 300, the foot 350 is set to be smaller by one size so that the apparatus has a compact configuration with only the size of the shield room main part 320 being distinct.

In this embodiment, because the pipe-like opening part 301 functions as the storage space of the measurement part 100 in the first state, the height H7 of the opening part 301 is set. However, when the opening part 301 may not function as the storage space, the height of the opening part 301 may be 840 mm which is the substantial height H9 of the opening part except for the height H8 (360 mm) of the plinth part 302.

However, the person to be measured has the sense of anxiety when the person to be measured is inserted into the opening part 301 that is small in the opening. Also, in order to remove the extrinsic magnetic field, there is required some degree of the depth L4 of the magnetic shield room 300. Therefore, in order to insert the person to be measured to a predetermined position of the magnetic shield room 300, the stage move mechanism part 220 having a long stroke is required.

Under the above circumstances, according to this embodiment, the opening part 301 is utilized as the retreat position of the measurement part 100 in the first state, and the height H10 is set to a height by which the stage 210 can be sufficiently received in the opening part 301 in the first state even if the stage 210 moves down, and the depth L5 by which the plinth part 302 is retreated. Then, the substantial height H9 and lateral width W5 of the opening part 301 have sufficient sizes to receive the stage 210 having the measurement part 100 at a predetermined position. This allows the above problem to be solved.

Also, in this embodiment, as shown in FIG. 9F, the inner part of the cover 324 is slanted. With this structure, since the opening of the opening part 301 can look more widely, the sense of anxiety of the person to be measured can be eased up.

In addition, in this embodiment, a holding implement 303 for holding the signal line 160 is disposed on the upper part of the other end side of the opening part 301 as shown in FIGS. 9A and 9E. With this structure, since the signal line 160 that is pulled out of the measurement part 100 backward of the opening part 301 is loosely held, the movement of the measurement part 100 in the direction of X can be smooth performed while reducing the disconnection of the signal line 160.

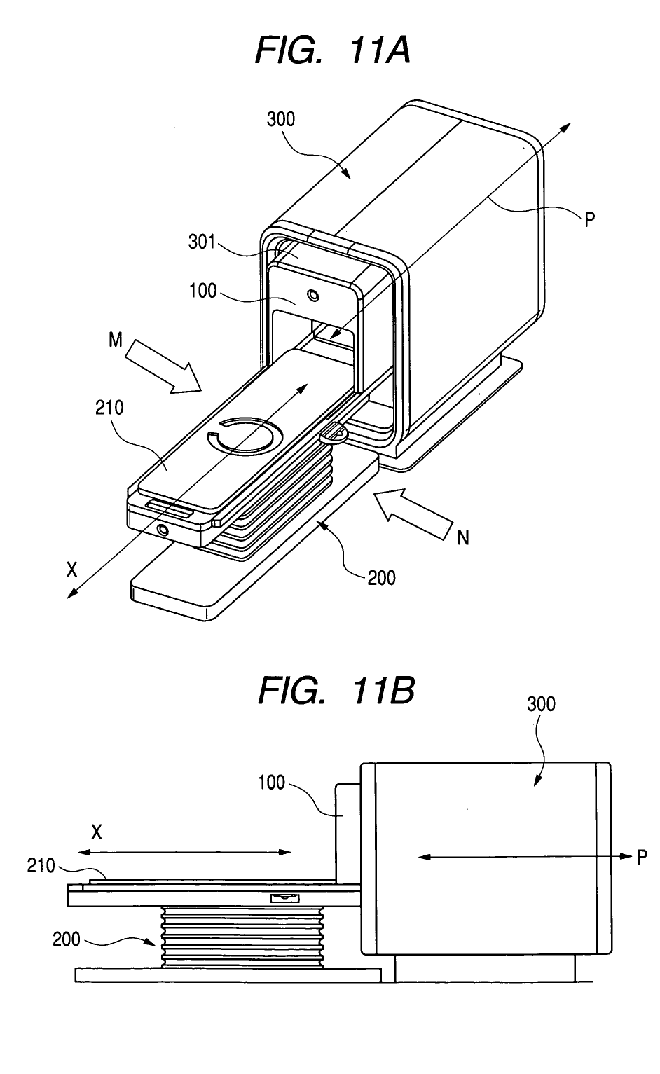

Then, the operation of the move mechanisms and the operation method will be described with reference to FIGS. 10 to 12B. FIG. 10 is a block diagram showing the circuit structure of the bed part. FIGS. 11A and 11B are external views showing a use state in which the bed part and the magnetic shield room are combined together, in which FIG. 11A is an external perspective view thereof, and FIG. 11B is a side view thereof. FIGS. 12A and 12B are reference views showing the use and operation state thereof, in which FIG. 12A is a cross-sectional view for explaining the operation of the first state to the second state, and FIG. 12B is a cross-sectional view showing the third state.

First, the device structure for the operation control of the bed part 200 will be described with reference to FIG. 10. The bed part 200 includes a control part 270 that controls the stage rise-and-fall mechanism part 240, the stage move mechanism part 220, the measurement department move mechanism part 230, and the gantry position adjustment mechanism part 150 that is made up of the direction adjustment mechanism part of Z 151 and the direction adjustment mechanism part of Y 152. The control part 270 is connected with a memory part 271 that stores the operation program of the plural move mechanism parts, the operation part 250 that operates those plural move mechanism parts, and a plurality of position sensors 272 that detect the operation positions of the plural mechanism parts. The bed part 200 also includes an oil supplier that supplies a hydraulic pressure to the move mechanism parts although being omitted from the description.

Also, the setting of the operation program of the control part 270 can be performed by the data collection analysis equipment 400.

Now, the operation of the heart magnetism measurement apparatus according to the operation of the operation part 250 shown in FIG. 6D will be described with reference to FIGS. 10 to 12B.

First, according to this embodiment, a stop state takes the first state shown in FIGS. 1A, 11B and indicated by a dotted line in FIG. 12A. In the first state, the bed part 200 and the magnetic shield room 300 are located linearly so that the longitudinal direction (direction of X) of the stage 210 and the main axis P of the pipe-like opening part 301 coincide with each other. Then, the measurement part 100 is received in the pipe-like opening part 301. The person to be measured uses the heart magnetism measurement apparatus from a direction of M, and the measurement engineer uses the heart magnetism measurement apparatus from a direction of N.

In the first state, the measurement engineer operates the down switch of the up-and-down switch 251 disposed on the operation part 250 so as to move down the stage 210 according to the height of the person to be measured.

As shown in FIGS. 12A and 12B, in this embodiment, the height H1 from the floor to the top face of the bed part 211 can be lowered downward from the reference value (home position) 715 mm of the first state by 315 mm, that is, the top face of the bed part 211 can be lowered to a position of 400 mm from the floor. The lowermost position is a height sufficient for an elder to sit at the top face of the bed part 211. The measurement engineer operates the up-and-down switch 251, sets an appropriate height, allows the person to be measured to sit at a place near the mark 218, urges the person to be measured to lie back with his head positioned at the measurement part 100 side, and can assist this action.

Then, the measurement engineer operates the up switch of the up-and-down switch 251 and then rises and falls the stage 210 to the reference value of the first state. In this situation, when the up switch is operated but the down switch is not operated, the control part 270 operates so as to rise and fall the stage 210 to the reference value. As a result, when the second state is shifted to the third state, the stage 210 can be prevented from colliding with the plinth part 302.

Also, when the up switch is operated to rise and fall the stage 210 to the reference value, the control part 270 stores a position at which the up switch is operated as a return position in the memory part 271.

Subsequently, the measurement engineer operates the move switch 252 so as to allow the measurement part 100 located at the retreat position to pass through the head of the person to be measured and move to the area of the heart. Since the move switch 252 has a pair of switches that allow the movement in the direction of X, those switches can be operated to position the measurement part 100 to the heart of the person to be measured. In this situation, since the operation part 250 is disposed between the mark 218 and the magnetic shield room 300, the measurement engineer can perform the positioning work by operating the operation part 250 by his right hand while looking through the opening part 109 of the measurement part 100. Moreover, since the position of the stage 210 is set to the height at which the measurement engineer who is standing is not forced to take an improper attitude, the workability of the workability of the measurement engineer can be improved.

In this embodiment, the move distance L6 of the measurement part 100 can be moved from the retreat position to the mark 218. As a result, the upper body of the person to be measured (position of from the head to the hip) can be covered. When the lower body of the person to be measured is measured, the person to be measured may be turned upside down and lie on the bed.

Then, the measurement engineer operates the pair of rise-and-fall switches 262 and the pair of move switches 263 in the adjustment switch country 260 so as to finely adjust the position of the sensors 102. First, in this fine adjustment, the rise-and-fall switch 262 is operated to fall the cooling container having the plurality of sensors 102 down to the area of the heart of the person to be measured. The lower end of the cooling container 101 is provided with the position sensors 272, and the position sensors 272 are set to generate a signal when there exists an obstruct at a predetermined position. In this embodiment, the position sensors 272 can be so set as to generate the signal at a first predetermined distance and a second predetermined distance.

For example, the control part 270 fixes the position of the measurement department move mechanism part 230 when the position sensor 272 generates the signal of the first predetermined distance. The control part 270 also stops the operation of the direction adjustment mechanism part of Z 151 when the position sensor 272 receives the signal of the second predetermined distance. In this embodiment, the first predetermined distance is set to 100 mm, and the second predetermined distance is set to 5 mm.

With this setting, since the position of the measurement department move mechanism part 230 is fixed at the time of the first predetermined distance, the cooling container 101 that has come down can be prevented from hitting the jaw of the person to be measured with the movement of the measurement part 100. Also, since the operation of the direction adjustment mechanism part of Z 151 stops at the time of the second predetermined distance, the cooling container 101 can be prevented from abutting against and putting pressure on the person to be measured.

In addition, the measurement engineer operates the move switch 263 so as to adjust the sensor position in the direction of Y. Then, the measurement engineer operates the lock switch 261 after the adjustment of the sensor position has been completed. The control part 270 fixes the positions of the stage rise-and-fall mechanism part 240, the measurement department move mechanism part 230 and the gantry position adjustment mechanism part 150 which have been operated up to now when the lock switch 261 is operated, and nullifies the operations from the switches that operate those mechanism parts. As a result, the erroneous operation can be prevented.

Then, the measurement engineer operates the move switch 253. When the move switch 253 is operated, the control part 270 operates the stage move mechanism part 220 and moves the stage 210 to a predetermined position of the magnetic shield room 300 from the state shown in FIG. 12A, as shown in FIG. 12B.

The measurement engineer can perform various measurements by operating the data collection analysis equipment 400. After the completion of those measurements, the measurement engineer operates the return switch 253. When the return switch 253 is operated, the control part 270 operates to return the stage 210 to the return position where the person to be measured gets on the stage 210. How to return and the operation order of the various move mechanism parts can be freely set. For example, first, the stage move mechanism part 220 is operated to change from the third state to the second state. In the second state, the gantry position adjustment mechanism part 150 is operated to return the gantry part 104 to a predetermined home position. Then, the measurement department move mechanism part 230 is operated to return the measurement part 100 to the predetermined home position from the second state to provide the first state. Then, the stage rise-and-fall mechanism part 240 is operated to return the measurement part to the return position. As other ways, it is possible to drive the move mechanism parts at the same time, or change the operation order of the various move mechanism parts. In addition, it is possible to return the measurement part from the third state to the first state. Those setting can be inputted from the data collection analysis equipment 400.

When the measurement engineer recognizes that the person to be measured gets off the bed part 200 at the return position and again operates the return switch 253, the control part 270 operates the stage rise-and-fall mechanism part 240 to return the bed part 200 to the first state.

As described above, the heart magnetism measurement apparatus according to this embodiment can provide the measurement engineer with the operation that is easy to measure by operating the plurality of mechanism parts through the operation part 250.

### Second Embodiment

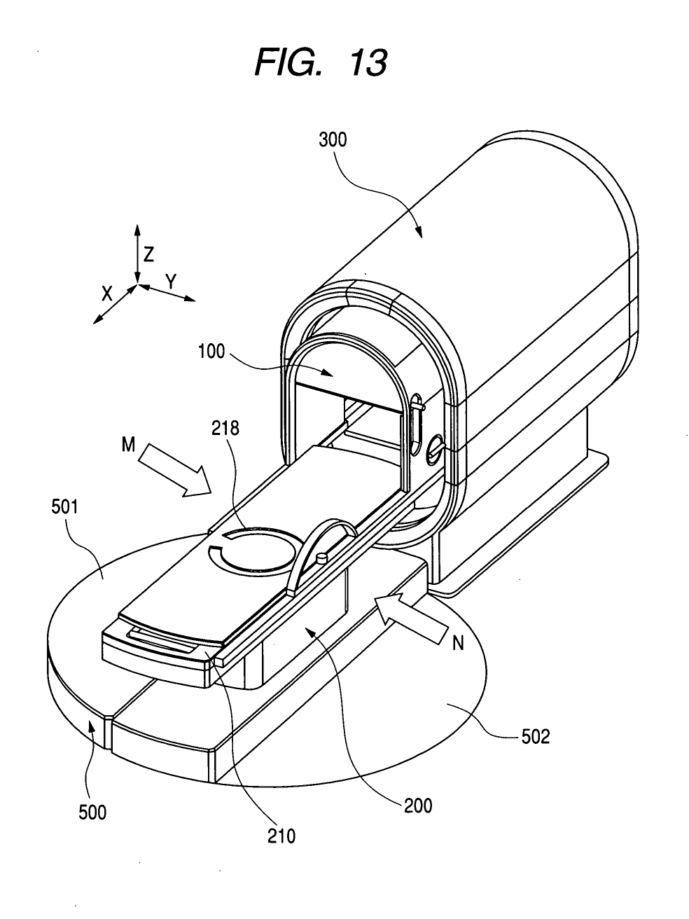

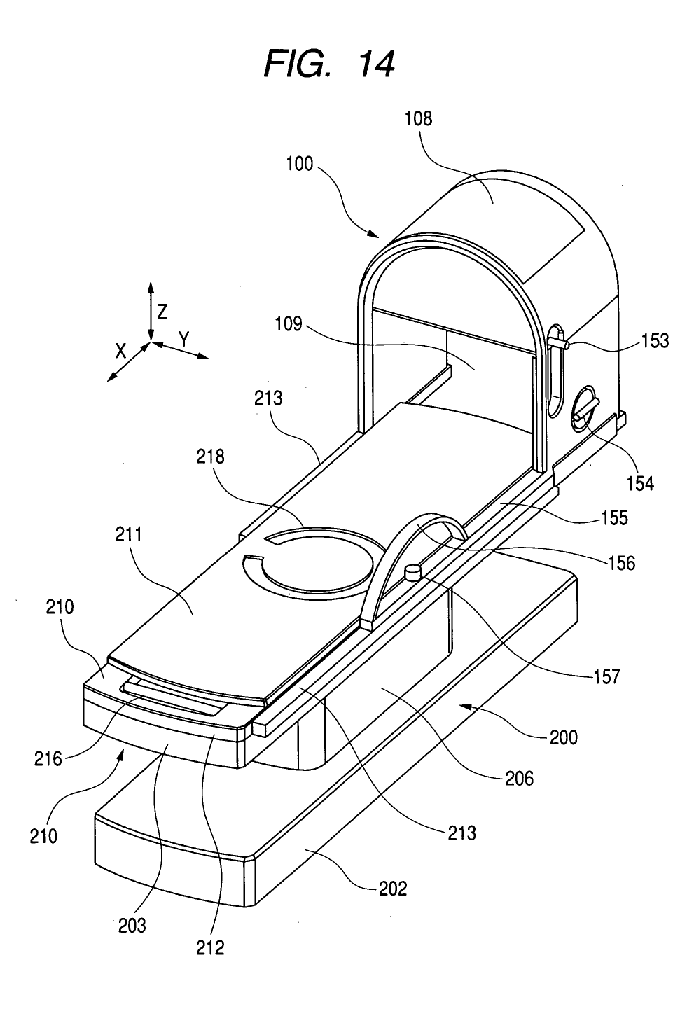

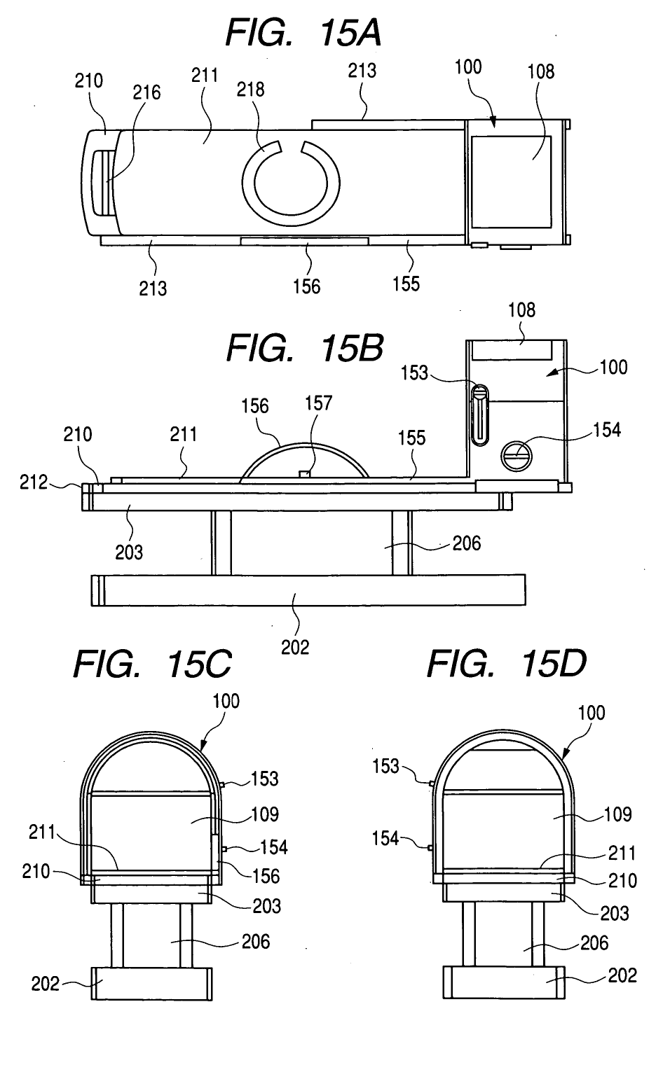

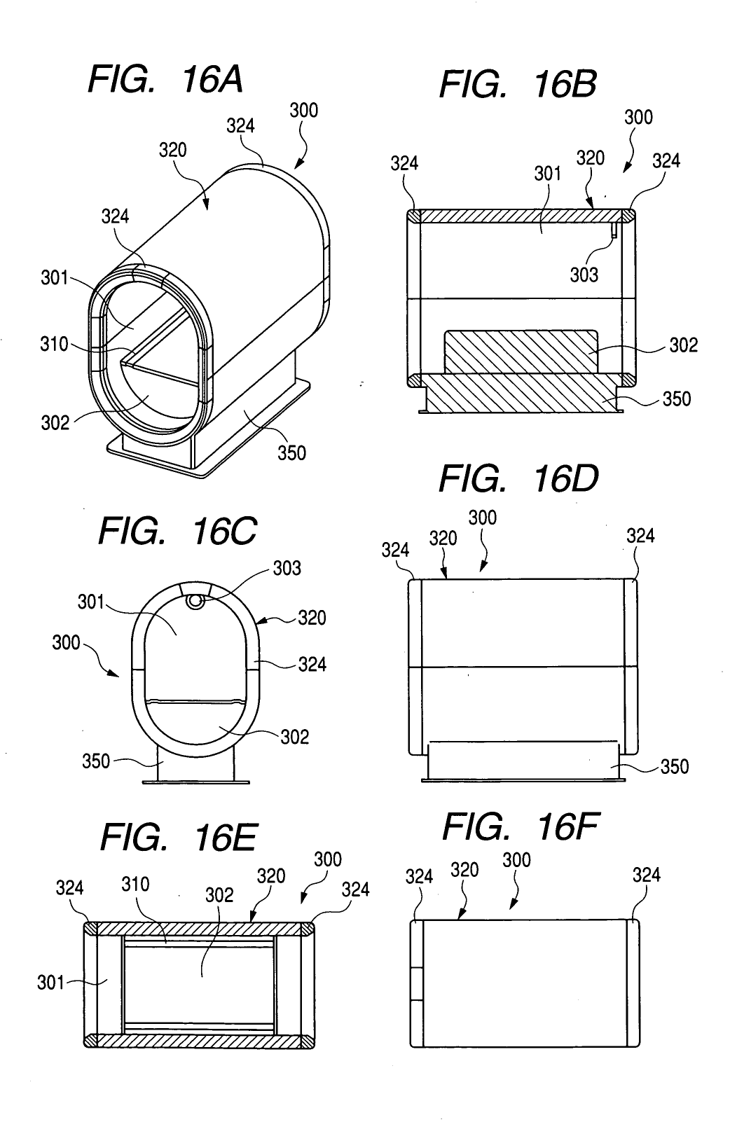

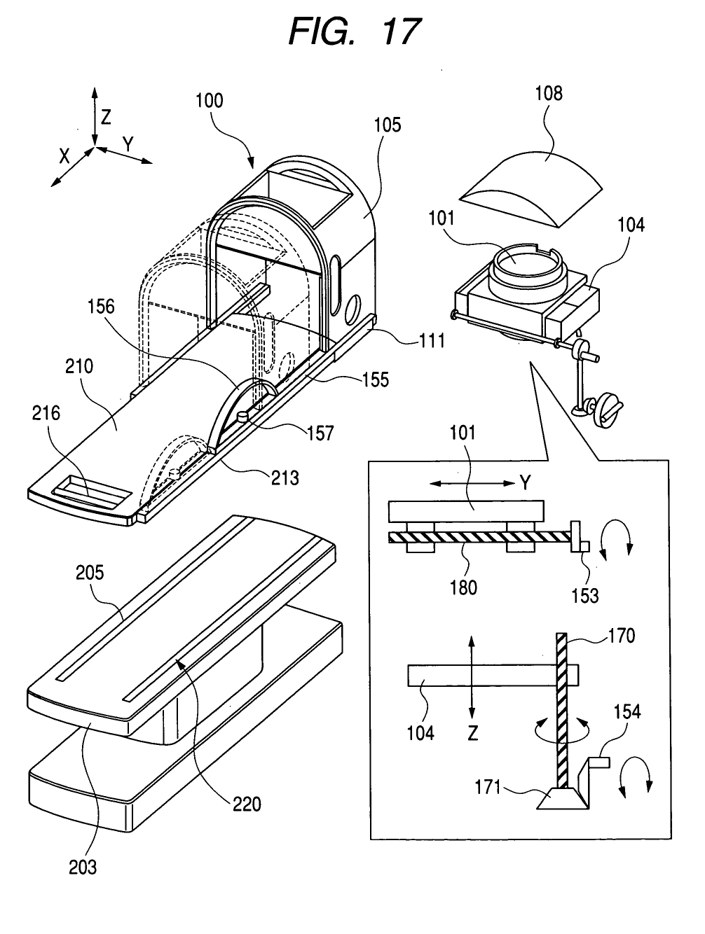

Subsequently, another embodiment of the present invention will be described with reference to FIGS. 13 to 17. FIG. 13 is an external view showing the use state of a heart magnetism measurement apparatus, FIG. 14 is an external perspective view of a bed part, FIGS. 15A to 15D are external views of the bed part, FIGS. 16A to 16F are external views of a magnetic shield room, and FIG. 17 is an explanatory diagram showing a movement mechanism part. The same structures or parts are indicated by like reference, and duplicate description will be omitted.

The outline structure of the heart magnetism measurement apparatus according to the second embodiment will be described with reference to FIG. 13. FIG. 13 shows the bed part 200, the magnetic shield room 300 and a mount table 500 of the bed part in the first state. The data collection analysis equipment 400 and the magnetic field measurement drive 500 have the same functions and arrangement as those in the first embodiment, and therefore will be omitted.

The heart magnetism measurement apparatus according to this embodiment are identical in the basic structure and arrangement with the heart magnetism measurement apparatus of the first embodiment, but is structured inexpensively.

One feature of the heart magnetism measurement apparatus according to the second embodiment resides in that the stage rise-and-fall mechanism part 240 is removed, and the stage 210 whose height is fixed is provided. In this embodiment, in order to improve ease to get on the stage 210 whose height is fixed, the mount table 500 is applied. The mount table 500 has a raised floor face 501 on one side of the bed part 200 in the direction of Y (direction side of M). The other side of the bed part 200 in the direction of Y (direction side of N) has a floor face 502 having the same height as that of the floor face on which the bed part 200 is located.

According to the mount table 500, the person to be measured can go up on the floor face raised from the normal floor face once, sit on the stage 210 from the floor face 501 and lie on the stage 210. On the other hand, the measurement engineer can conduct care of the person to be measured and operation on the same floor face as the installation face of the bed part 200. Accordingly, the person to be measured needs to go up on the floor face 501 once, but the same advantages as those in the stage rise-and-fall mechanism part 240 can be obtained. On the other hand, since the height of the measurement engineer is identical with the installation position of the bed part 200, the same advantages as those in the first embodiment can be obtained.

Also, in this embodiment, the mount table 500 is formed circularly with the mark 218 as a center thereof when being viewed from above. Therefore, the person to be measured can go up on the floor face 501 from any position in the direction of M. According to the circular shape, the advantage that the person to be measured is led toward the mark 218 which is the center of the circle can be expected.

Also, another feature of this embodiment resides in that the stage move mechanism part 220, the measurement department move mechanism part 230 and the gantry position adjustment mechanism part 150 consisting of the direction adjustment mechanism part of Z 151 and the direction adjustment mechanism part of Y 152 are manually operated. The manual structure makes it possible to remarkably reduce the costs.

Also, still another feature of this embodiment resides in that the stage rise-and-fall mechanism part 240 is removed, to thereby make the external configuration of the magnetic shield room 300 compact. In this embodiment, the downsized and compact external image can be greatly impressed due to the external configuration whose cross section is rounded like a track.

Hereinafter, the heart magnetism measurement apparatus according to the second embodiment will be described in more detail with reference to FIGS. 14 to 17.

First, the external structure of the bed part will be described with reference to FIGS. 14, 15A to 15D and 17. FIG. 14 is a perspective view of the bed part. FIGS. 15A to 15D are external views of the bed part, in which FIG. 15A is a plan view thereof, FIG. 15B is a front view thereof, FIG. 15C is a left side view thereof, and FIG. 15D is a right side view thereof. FIG. 17 is an explanatory diagram of the move mechanism part.

Referring to FIGS. 14 and 15A to 15D, in this embodiment, the bed part 200 is made up of the basis 201 located on the floor, the stage 210 disposed on an upper part of the basis 201 so as to be slidable in the direction of X, and the measurement part 100 disposed on one end side of the stage 210 in a longitudinal direction thereof. The basis 201 is made up of a foot 202 located on the floor, the stage support part 203 that supports the stage 210, and a support column part 206 that connects the foot 202 and the stage support part 203.

As shown in FIG. 17, the stage move mechanism part 220 consisting of a pair of support rails 205 that is disposed in the direction of X is disposed on the top face of the stage support part 203 so that the stage 210 can be moved in the direction of X.

Returning to FIGS. 14 and 15A to 15D, the support column part 206 is so formed as to be smaller than the stage support part 203 and the foot 202 by one size, and a lower limb space is broadly set. Because the height of the foot 202 is set to the same height as that of the floor face 501 of the mount table 500, the person to be measured gets such an impression that the stage 210 floats, and the broad lower limb space is further impressed.

The stage 210 has the same structure as that in the first embodiment, that is, includes the frame component 212, the bed 211 and the measurement department support rail 213. Slide rails of the stage support part 203 as well as slide rails that constitute the stage move mechanism part 220 are disposed on the lower face of the frame component 212 so as to be slidable in the direction of X.

Lock slots not shown which are divided into a plurality of pieces are defined at given intervals on one side of the measurement department support rails 213 so that the movement of the measurement part 100 in the direction of X can be locked at the given intervals.

In addition, a handle 216 is disposed on one end side of the stage 210, and the measurement part 210 is disposed on the other end side of the stage 210 opposite to the handle 216 in the direction of X. In this embodiment, since the stage move mechanism part 220 is manually operated, the stage 210 is moved in the direction of X by means of the handle 216. The handle 216 has a lock button not shown and can fix the movement of the stage 210 at two places, that is, the first state shown in FIG. 14 and the third state shown in FIG. 2.

The measurement part 100 has an upper part shaped in a circular arc when being viewed from the direction of X. The semicircular part of the upper part of the measurement part 100 is flat when being viewed from the direction of X, and the lower part of the measurement part 100 is formed by the opening part 109 that is pierced in the direction of X. The gantry 104 having the cooling container 101 is movably supported on the semicircular part of the measurement part 100 by the manual gantry position adjustment mechanism part 150.

In this embodiment, since the size of the upper end of the cooling container 101 is set to be smaller than the lateral width of the measurement part 100, the upper external configuration of the measurement part 100 can be shaped in the circular arc.

A direction rotation lever of Y 153 that operates the manual direction adjustment mechanism part of Y 152 and a direction rotation lever of Z 154 that operates the manual direction adjustment mechanism part of Z 151 are disposed on one side plate of the measurement part 100.

The above structure will be described in more detail with reference to FIG. 17. The gantry 104 is supported by a plurality of screw bars 170 disposed on the measurement department main part 105. The screw bars 170 are arranged in such a manner that the longitudinal direction thereof coincides with the direction of Z, and a trapezoidal gear 171 is disposed on the lower end of the screw bars 170. The rotation lever 154 of the direction of Z is equipped with a trapezoidal gear that is meshed with the gear 171. With the rotation of the rotation lever 154 of the direction of Z, the rotation force is transmitted to the gear 171, thereby making it possible to rotate the screw bar 170. The gantry 104 has a screw that is meshed with the screw bar 170, and the gantry 104 can rise and fall due to the rotation of the screw bar 170.

On the other hand, the gantry 104 is equipped with a screw bar 180 whose longitudinal direction is arranged along the direction of Y. A rotation level 153 of the direction of Y is disposed on one end side of the screw bar 180, and also the cooling container 101 having a screw that is meshed with the screw bar 180 is disposed on the one end side of the screw bar 180. With this structure, the rotation lever 153 is rotated, thereby making it possible to move the cooling container 101 in the direction of Y.

In this embodiment, since the rotation lever 153 of the direction of Y rises and falls while the gantry 104 rises and falls, the rotation lever 153 is operated through a window longer in the direction of Z.

In addition, a handle support stick 155 that extends along the measurement department support rail 213 is disposed on one side plate of the measurement part 100. The handle support stick 155 moves on the measurement department support rail 213 while the measurement part 100 moves. An arch handle 156 is disposed on an end of the handle support stick 155. The measurement engineer grips and moves the arch handle 156 in the direction of X, thereby making it possible to move the measurement part 100 in the direction of X.

Then, the handle 156 is so disposed as to be positioned on one side of the mark 218 in the first state of FIG. 15. Accordingly, in the first state, since the handle 156 is positioned in the vicinity of the mark 218, the handle 156 can be used as a hand rail when the person to be measured lies on the bed. On the other hand, the measurement engineer can grip and move the handle 156 from the first state to the second state.

In the second state indicated by a dotted line in FIG. 17, since the handle 156 moves to the end of the stage 210, it causes no trouble in the positioning operation of the gantry position adjustment mechanism part 150. Also, since the handle 156 is positioned in the vicinity of the handle 216 that is disposed on the stage 210 in the second state, the handle 156 can also function as a handle for moving to the third state.

Also, in this embodiment, rail holding parts 111 are disposed on both lower ends of the measurement part 100 and attached so as to interpose the measurement department support rail 213 from both sides thereof. With this structure, the measurement part 100 is prevented from being derailed from the measurement department support rail 213.

Also, a lock switch 157 is disposed on the lower part of the arch handle 156. The lock switch 157 engages with the lock slots defined in the measurement department support rail 213 so as to fix the measurement part 100 to a predetermined position.

Subsequently, the external structure of the magnetic shield room 300 will be described with reference to FIGS. 16A to 16F. FIGS. 16A to 16F are external views of the magnetic shield room, in which FIG. 16A is a perspective view thereof, FIG. 16B is a vertical cross-sectional view thereof, FIG. 16C is a left side view thereof, FIG. 16D is a front view thereof, FIG. 16E is a horizontal cross-sectional view thereof, and FIG. 16F is a top view thereof.

The magnetic shield room 300 according to the second embodiment is largely different from that of the first embodiment in that the external configuration viewed from the direction of X shown in FIG. 16C is shaped in a track, and other portions are identical in the structure with those in the first embodiment.

In this embodiment, because the stage 210 of the bed part 200 does not rise and fall, the lateral width W5 of the top face of the plinth part 302 which supports the lateral width W0 of the stage 210 is not required for a position lower than the plinth part 302. For that reason, in this embodiment, the configuration of the opening part 301 below the top face of the plinth part 302 is shaped in a circular arc. Similarly, the upper part of the opening part 301 is shaped in a circular arc in correspondence with the circular arc shape of the upper part of the measurement part 100. Accordingly, in this embodiment, since the pipe-like opening part 301 is shaped in a track, the appearance of the magnetic shield room 300 is also consequently in the shape of a track-like pipe in the cross section. Other portions are identical with those in the first embodiment, and their description will be omitted.

As was described above, since the heart magnetism measurement apparatus according to the second embodiment can move the various move mechanism parts by manual operation, the manufacture costs can be largely suppressed. In particular, since the stage rise-and-fall mechanism part 240 is operated manually to make the magnetic shield room 300 compact, the further downsized apparatus can be realized.

In the second embodiment, it is possible that only the stage rise-and-fall mechanism part 240 is operated manually, and other move mechanism parts are operated automatically as in the first embodiment. According to this structure, since the automation can be promoted with the compact appearance, a work of the measurement engineer can be reduced.

1. A magnetic field measurement apparatus, comprising:
a basis fixed to a floor;
a stage on which a person to be measured gets;
a measurement part having a plurality of flux meters;
a stage move mechanism part that moves the stage with respect to the basis in a longitudinal direction of the basis; and
a measurement department move mechanism part that moves the measurement part with respect to the stage in a longitudinal direction of the stage,
wherein the apparatus takes a first state where the measurement part is retreated to one side of the stage in the longitudinal direction through the measurement department move mechanism part, a second state where the measurement part is moved from the first state in the longitudinal direction and fixed to a predetermined position through the measurement department move mechanism part, and a third state where the stage is moved from the second state while keeping a positional relationship between the stage and the measurement part, and fixed to a predetermined position through the stage move mechanism part.

2. The magnetic field measurement apparatus according to claim 1, wherein the stage includes a pair of measurement department support rails that support the measurement part at both sides of the stage in the widthwise direction, and
wherein the measurement part includes a pair of side plates that are supported by the measurement department support rails, and an opening part through which the person to be measured passes while the measurement part moves, the opening part being disposed between the pair of side plates.

3. The magnetic field measurement apparatus according to claim 2, wherein the measurement part includes a flux meter position adjustment mechanism part that adjusts the positions of the plurality of flux meters.

4. The magnetic field measurement apparatus according to claim 3, wherein the flux meter position adjustment mechanism part includes a drive means for driving the plurality of flux meters in a heightwise direction, a control means for controlling the drive means, and a detecting means for detecting the positions of the plurality of flux meters in the heightwise direction, and
wherein the control means suspends the drive means at a first detection position, and stops the drive means at a second detection position that is lower than the first detection position.

5. The magnetic field measurement apparatus according to claim 1, further comprising a pipe-like magnetic shield room having an opening part whose main axis coincides with an extension of the stage in the longitudinal direction,
wherein the magnetic shield room side is at a retreat position of the measurement part in the first state, and
wherein the predetermined position in the third state is within the magnetic shield room.

6. The magnetic field measurement apparatus according to claim 5, wherein the retreat position of the measurement part in the first state is within the magnetic shield room, and
wherein a support means that supports one end of the stage in the third state is disposed within the pipe-like opening part.

7. The magnetic field measurement apparatus according to claim 6, wherein the basis includes a stage rise-and-fall mechanism part that rises and falls the stage, and
wherein the pipe-like opening part has a size that enables the operation of the stage rise-and-fall mechanism part in the first state.

8. A magnetic field measurement apparatus, comprising:
a bed part having a stage on which a person to be measured lies on a top face of the bed part; and
a pipe-like magnetic shield room that is located on a floor in a predetermined positional relationship with the bed part,
wherein the bed part includes a measurement department move mechanism part that moves a measurement part disposed on one end side of the stage in a longitudinal direction of the stage toward a center of the stage along the longitudinal direction, and a stage move mechanism part that moves the measurement part that has moved to the center of the stage to an interior of the magnetic shield room together with the stage.

9. A magnetic field measurement apparatus, comprising:
a bed part having a stage on which a person to be measured lies on a top face of the bed part; and
a pipe-like magnetic shield room that has a main axis that coincides with an extension of the stage in a longitudinal direction of the stage,
wherein the bed part has a stage that moves in the longitudinal direction through a first move mechanism part, and a measurement part on the stage which moves in the longitudinal direction through a second move mechanism part,
wherein the apparatus takes a first state where the measurement part is retreated to a magnetic shield room side of the stage in the longitudinal direction, a second state where the measurement part is moved from the first state to a measurement position, and a third state where the stage is received at a predetermined position within the magnetic shield room while keeping a positional relationship between the stage and the measurement part in the second state.

10. The magnetic field measurement apparatus according to claim 9, wherein a retreat position of the measurement part in the first state is within the magnetic shield room, and
wherein a support means that supports one end of the stage in the third state is disposed within the pipe-like opening part.

11. The magnetic field measurement apparatus according to claim 10, wherein the stage has a pair of measurement department support rails that support the measurement part at both sides of the stage in the widthwise direction of the stage, and
wherein the measurement part includes a pair of side plates that are supported by the measurement department support rails, and an opening part through which the person to be measured passes while the measurement part moves, the opening part being disposed between the pair of side plates.

12. The magnetic field measurement apparatus according to claim 11, wherein the measurement part includes a flux meter position adjustment mechanism part that adjusts the positions of a plurality of flux meters.

13. The magnetic field measurement apparatus according to claim 12, wherein the flux meter position adjustment mechanism part includes a drive means for driving the plurality of flux meters in a heightwise direction, a control means for controlling the drive means, and a detecting means for detecting the positions of the plurality of flux meters in the heightwise direction, and
wherein the control means suspends the drive means at a first detection position, and stops the drive means at a second detection position that is lower than the first detection position.

14. The magnetic field measurement apparatus according to claim 10, wherein a basis includes a stage rise-and-fall mechanism part that rises and falls the stage, and
wherein the pipe-like opening part has a size that enables the operation of the stage rise-and-fall mechanism part in the first state.
